(19) United States
(12) Patent Application Publication
(10) Pub. No.: US 2014/0255465 A1
(43) Pub. Date: Sep. 11, 2014

(54) Process For The Preparation Of A Non-Corrosive Base Solution And Methods Of Using Same

(71) Applicants: Suzannah Jane ROBERTS, Bellevue, WA (US); Stephanie Jo BLACKWELL, Kirkland, WA (US); Shannon Joe BROWN, Grass Valley, CA (US)

(72) Inventors: Suzannah Jane ROBERTS, Bellevue, WA (US); Stephanie Jo BLACKWELL, Kirkland, WA (US); Shannon Joe BROWN, Grass Valley, CA (US)

(73) Assignee: COGNATE3 LLC, Bellevue, WA (US)

(21) Appl. No.: 14/201,864

(22) Filed: Mar. 9, 2014

Related U.S. Application Data
(60) Provisional application No. 61/774,622, filed on Mar. 8, 2013.

(51) Int. Cl. A61K 33/08

(52) U.S. Cl. CPC A61K 33/08
USPC 424/447; 424/688

(57) ABSTRACT

The present invention provides novel methods of making a non-corrosive base solution for use as an alkalinity increasing agent and/or antioxidant. The present invention further provides novel compositions and methods which can be used to provide relief from acidosis, acidemia, and disorders related to or complicated by acidosis or excessive free radical or other reactive oxygen species production in mammalian subjects.

### RELATED APPLICATIONS

This application claims priority benefit of U.S. Provisional patent application Ser. No. 61/774,622, filed Mar. 8, 2013 and U.S. Provisional patent application Ser. No. 61/774,626, filed Mar. 8, 2013, the disclosure of each which is incorporated herein in its entirety by reference.

### TECHNICAL FIELD

The present invention relates to methods of making a non-corrosive base solution and the use of the non-corrosive base solution in mammalian subjects. More specifically, the present invention relates to methods and compositions for altering pH levels in mammalian subjects.

### ADDITIONAL DISCLOSURE

Additional disclosures relating to the instant application may be found in U.S. Provisional patent application Ser. No. 60/947,633, filed Jul. 2, 2007, U.S. patent application Ser. No. 12/167,123, filed Jul. 2, 2008, now U.S. Pat. No. 8,273,384, issued Sep. 25, 2012, and U.S. Provisional patent application 61/757,059, filed Jan. 25, 2013, each of which is incorporated herein by reference in its entirety for all purposes.

### BACKGROUND

The pH, or hydrogen ion concentration, [H+], is a logarithmic measurement of the concentration of hydrogen in an aqueous solution. The lower the pH is, the higher the concentration of hydrogen ion. Conversely, the higher the pH is, the lower the concentration of hydrogen ion. The pH of natural environments varies from about 0.5 in the most acidic soils to about 10.5 in the most alkaline lakes with most environments falling somewhere in between. The range of pH over which an organism grows is defined by the minimum pH, below which the organism cannot grow or reproduce; the maximum pH, above which the organism cannot grow or reproduce; and the optimum pH, at which the organism grows best. For most organisms there is an orderly increase in growth rate and reproduction rate between the minimum and the optimum pH and a corresponding orderly decrease in growth rate and reproduction rate between the optimum and the maximum pH, reflecting the general effect of changing [H+] on the rates of enzymatic reaction.

Under optimal conditions, the human body maintains very narrow pH ranges. The optimal pH of blood in humans is maintained between a pH 7.3 to 7.4. For optimal health, the pH of blood should be about 7.365. The pH of urine can vary between 4.6 and 8, with a pH of 7 being norm and high levels of uric acid, i.e. a lower pH, indicating possible kidney disease, cancer, alcoholism, liver disease, and lipid disorders among others. The pH of saliva is generally between 6.0 and 7.4 and increased acidity may be an indication of a diet high in acidic or sugary foods. An alteration in the optimal pH of any of these fluids may make an individual more susceptible to infection and disease by creating a more hospitable environment for microorganisms to grow.

The human body attempts to maintain optimal pH through the actions of buffers, respiration, and renal function. In dealing with the normal acid load from diet and metabolism, buffers such as proteins, phosphate, and H₂CO₃:HCO₃— act to control the pH level. Respiration maintains a constant carbonic acid level at 1.2 meq/l or PaCO₂ of 40 mmHG through either excretion or retention of CO₂ by the lungs. Respiration can also rapidly compensate for changes in pH by altering the level of PaCO₂ through the alteration of alveolar ventilation. The renal system manipulates the volume and composition of extracellular fluid to help maintain the pH of plasma. However, while the renal system can correct states of excess, it cannot correct states of deficiency such as through loss of Na+, K+ or HCO₃⁻. Additionally, unlike respiratory regulation, regulation of pH through renal function can take several days.

pH levels can be affected by a number of exterior factors including, stress, diet, exposure to toxins, poor sleeping habits, and exercise. During acute stress, the heart rate and arterial blood pressure are increased, demand for oxygen is increased and lactic acid levels in the blood can rise by as much as 47% (Vrijikotte, 2000; and Kubera, 2012). All of these events can contribute to a change in the pH of plasma. The Western diet can also affect pH levels. As an individual consumes more animal-based foods compared to plant-based foods, diets become more acid producing. (Ströhle, 2010) In the case of toxin exposure, individuals may have both metabolic and respiratory acidosis. Metabolic acidosis leads to alveolar hyperventilation with a fall in PaCO₂ (partial pressure in CO₂). Toxin exposure can also decrease breathing rates leading to a decrease in the rate of CO₂ expulsion from the lungs and respiratory acidosis. Sleep apnea can lead to elevated PaCO₂ (>45 mm Hg) with acidemia (i.e., pH<7.35) (Bizzel, 2009).

Even healthy pursuits can affect pH levels. As people exercise, heart rate, systolic blood pressure, and cardiac output increase. The body's metabolism becomes more active, producing CO₂ and H⁺, and respiration increases to compensate for increased oxygen demand. Eventually, an individual's metabolism exceeds the body's oxygen supply and the body uses the lactic acid system (anaerobic glycolysis) to generate energy. These chemical changes can cause the pH of the blood to drop and (H+) to accumulate (Robergs, Ghiasvand, Parker 2004). The normal pH of the muscle cell is 7.1 but if the buildup of H+ continues and pH is reduced to around 6.5 then muscle contraction may be impaired. (Garrett, 2000) When an individual's exercise intensity is sufficient to cross the lactic threshold. i.e. there is an abrupt increase in blood lactate levels, exercise becomes more difficult. (Roberts & Roberg, 1997). Muscles ache and burn and become extremely fatigued. Symptoms increase if the exercise continues making it difficult if not impossible to maintain exercise levels at the desired intensity.

Failure or overloading of any of the body's normal regulatory mechanisms, whether through stress, pharmacological treatments, diet, or disease can cause acidosis, impacting an individual's wellbeing and quality of life and leaving them more susceptible to infection and conditions such as cancer, cardiovascular disease, fibromyalgia, hepatic disease, gout, arthritis, anemia, sepsis, weight gain, staph infections including methicillin-resistant Staphylococcus aureus, streptococcus, systemic inflammatory response syndrome, gout, arthritis, sepsis, diabetic neuropathy, confusion, diabetes, cellulitis and pancreatic impairment. There is therefore a need for compositions that can compensate for the failure of regulatory mechanisms to maintain physiological pH1 and prevent acidosis.

### SUMMARY

Provided herein are compositions and methods for maintaining optimal pH and treating or preventing acidosis. Compositions and methods as described herein may further be used in the treatment of conditions caused or exacerbated by acidosis, specifically lactic acidosis.

Acidosis may be accompanied by the buildup of lactate, particularly D-lactate. This buildup of lactate generally occurs when cells are hypoxic and functioning anaerobically. Impaired cellular respiration leads to lower pH levels and can be indicative of tissue hypoxia, hypoperfusion and possible damage. Compositions and methods of the present invention increase pH levels, preventing or treating hyperlactemia (lactate concentrations between 2 mmol/L and 5 mmol/L) and lactic acidosis (lactate levels>5 mmol/L and serum pH<7.35).

Compositions and methods as described herein may additionally be used for optimizing health and performance; preventing illness; decreasing recovery times from exertion, illness, and injury; increasing energy levels; improving exercise performance; improving hydration; preventing muscle damage after exercise; and increasing stamina during exercise. Compositions and methods described herein may further be used in the treatment and prevention of conditions caused by or exacerbated by having acidosis such as infections, cancer, arthritis, gout, sepsis and diabetes.

Compositions and methods as described herein may treat or prevent acidosis by a variety of means. In some embodiments, the compositions and methods of the present invention may decrease lactate levels, specifically D-lactate. The normal blood lactate concentration is 0.5-1 mmol/L. Individuals in various disease states may have lactate concentrations of less than 2 mmol/L. Hyperlactemia is defined as a mild-to-moderate persistent increasing blood lactate concentration (2-5 mmol/L) without metabolic acidosis, whereas lactic acidosis is characterized by persistently increased blood lactate levels (usually >4-5 mmol/L) in association with metabolic acidosis. The compositions and methods described herein are useful in the treatment and prevention of both hyperlactemia and lactic acidosis.

The normal pH of intracellular and interstitial fluids is maintained because acids are removed at the same rate they are added. If acid is added faster than it is removed, the pH of intracellular and interstitial fluids decreases, resulting in acidosis. While strong mineral bases have often been used to neutralize acids, they are very corrosive and are not generally suitable for altering pH in living organisms. The present invention provides compositions and methods for altering base solutions so that they may be effectively used to increase pH levels in living organisms.

Reactive oxygen species including free radicals are produced as part of the normal metabolic process. They are generally prevented from causing damage through enzymes such as superoxide dismutases and catalases as well as antioxidants or other free radical scavengers. When levels of ROS exceed the neutralizing capacity of the body's regulatory mechanisms, such as during periods of intense exercise or due to a failure in endogenous antioxidant production, cellular damage, mutation and/or mortality levels increase. Proton absorbers which have previously been used to reduce free radicals are not true bases and require consumption of such large amounts that they are not generally suitable for prolonged use.

The methods and formulations of the present invention provide a base solution (Alkaline water) with a high concentration of OH⁻ ions which may be used as an alkalinity increasing agent, an antioxidant and/or free radical scavenger. In some embodiments, the methods of the present invention combine a magnetically treated calcium hydroxide solution with an ozone treated sulfuric acid solution to create such a base solution with a high concentration of OH⁻ ions. The alkaline water described herein may be taken internally and/or applied topically in dermatological formulations.

Alkaline water as described herein may be manufactured by whatever means useful to create a water with a pH between about 7 to about 14, preferably a pH of about 7.5 to about 12.75, preferably a pH of about 10 to about 11, about 12 to about 14, about 12.25 to about 12.75, more preferably about 12.3 to about 13.8, preferably a pH of about 12.5 to about 13.75.

In some embodiments, alkaline water may be manufactured by combining oxides including, but not limited to, calcium hydroxide, calcium oxide, sodium hydroxide, sodium oxide, potassium hydroxide, potassium oxide, magnesium hydroxide, or magnesium oxide, with mineral free water to create a non-corrosive base solution with a high concentration of OH⁻ ions.

In some embodiments, the solution containing the oxide or hydroxide and water is stirred to increase a rate or amount of dissociation of the OH⁻ ions. After mixing, the ionic concentration in the water will result in a conductivity measurement of between about 50 μS/cm to about 2000 μS/cm, preferably between about 100 μS/cm to about 1000 μS/cm, preferably about 500 μS/cm to about 800 μS/cm, preferably about 650 μS/cm to about 750 μS/cm, preferably about 700 μS/cm to about 2000 μS/cm, preferably about 700 μS/cm to about 750 μS/cm.

The resulting concentrated alkaline water may be consumed directly or diluted with filtered water to a pH of about 7 to about 10, about 7.5 to about 8. The diluted alkaline water will have a conductivity of about 45 μS/cm to about 90 μS/cm, about 50 μS/cm to about 75 μS/cm, about 50 μS/cm to about 60 μS/cm. In some embodiments, the concentrated alkaline water may be used to make a gel for topical administration.

Calcium hydroxide (Ca(OH)₂) is a base which may be used to create alkaline water for use as an acid neutralizing agent and/or antioxidant as described herein. However, it will only dissociate slightly in a weak acid environment. At a pH of 5.5 or higher, calcium hydroxide rapidly loses its solubility and at a pH of 8.0 it is insoluble. In one embodiment, the present invention provides a method of increasing the solubility of calcium hydroxide allowing a larger volume of Ca(OH)₂ to be dissociated in solution including weakly acidic, neutral or slightly basic solutions. In a further embodiment, the present invention provides a means of raising the pH of a Ca(OH)₂ solution at least one pH point higher than a normal saturated calcium hydroxide solution. In some embodiments, the present invention provides a method of increasing the reactivity of Ca(OH)₂ in solution. In another embodiment, the present invention provides a method for increasing the level of free hydroxide in a solution made with Ca(OH)₂ through the removal of calcium ions.

Useful forms of calcium hydroxide for use within the formulations and methods of the invention include the forms described herein, as well as solvates, hydrates, or combinations thereof.

Sulfuric acid is a strong mineral acid. In the compositions and methods of the present invention, sulfuric acid in water is treated to reduce the acidity while maintaining the concentration of sulfate in the solution. Such treatment may be accomplished by any means possible, including the addition of oxygen to the sulfuric acid solution. In some embodiments, the sulfuric acid solution is infused with ozone. Such treatments may increase the pH of the sulfuric acid solution, creating a neutral or basic solution which may then be combined with the calcium hydroxide solution described above to create a non-corrosive base solution with a high concentration of OH⁻ ions.

In another embodiment, calcium oxide (CaO), another strong base, is used to form Alkaline water to be used as an acid neutralizing agent and/or antioxidant as described herein. Calcium oxide is stirred into purified, distilled, spring, filtered, or mineral free water to create a non-corrosive base solution with a high concentration of OH⁻ ions. Such a solution will have a pH between about 7 to about 14, preferably a pH of about 12 to about 14, more preferably about 12.3 to about 13.8, preferably a pH of about 12.5 to about 13.75 and an ionic concentration such that the conductivity would be about 50 μS/cm to about 2000 μS/cm, preferably between about 100 μS/cm to about 1000 μS/cm, preferably about 500 μS/cm to about 800 μS/cm, preferably about 650 μS/cm to about 750 μS/cm, preferably about 700 μS/cm to about 750 μS/cm. In some embodiments the ionic concentration is such that the conductivity may be between about 700 μS/cm to about 2000 μS/cm.

The resulting concentrated alkaline water may be consumed directly or diluted with filtered water to a pH of about 7 to about 10, about 7.5 to about 8. The diluted alkaline water may have a conductivity of about 45 μS/cm to about 90 μS/cm, about 50 μS/cm to about 75 μS/cm, about 50 μS/cm to about 60 μS/cm.

In exemplary embodiments, the compositions and methods described herein employ a base solution (also referred to as an OH⁻ solution or Alkaline water) as described above to increase or maintain physiological pH in a mammalian subject.

Mammalian subjects amenable for treatment according to the formulations and methods of the invention include, but are not limited to, human and other mammalian subjects with acidosis, as well as conditions associated with or complicated by acidosis including, but not limited to, methicillin resistant staphylococcus aureus (MRSA), streptococcus infections, sepsis, systemic inflammatory response syndrome (SIRS), folliculitis, gout, arthritis, hypoxia, hypoperfusion, hemorrhage, ethanol toxicity, shock, hepatic disease, diabetic ketoacidosis, exercise fatigue, non-Hodgkin's and Burkitt's lymphoma, systemic inflammatory response syndrome (SIRS), hyperventilation, abdominal pain, lethargy, shock, severe anemia, hypotension, irregular heart rhythm, tachycardia, fibromyalgia, weight gain, cancer, cardiovascular disease, respiratory disease, infection, diabetes, diabetic neuropathy, cellulitis and pancreatic impairment. In other embodiments, the compositions and methods of the present invention are used as anti-bacterial agents.

Mammalian subjects amenable for treatment according to the formulations and methods of the invention further include, but are not limited to, human and other mammalian subjects with symptoms of acidosis, as well as symptoms or conditions associated with or complicated by acidosis. Such symptoms include, but are not limited to, acidemia, extreme tenderness in the joint, inflammation, swelling, pain, redness in the affected area, confusion, lethargy, rapid breathing, shortness of breath, wheezing, chest pain or pressure, joint stiffness, swelling, joint deformity, crepitus, non-specific fever, joint inflammation, headaches, fatigue, constipation, a feeling of euphoria, nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, easy bruising, vomiting, ascites, dry skin, dry mouth, low blood pressure, frequent urination, chest pain, lymph node pain, night sweats, skin rash, hyperventilation, abdominal pain, severe anemia, musculoskeletal pain, memory issues, and light headedness.

In some embodiments, the compositions and methods described herein may be used to speed recovery times, particularly from periods of stress or intense physical activity. In other embodiments, the compositions and methods described herein may be used to increase endurance, decrease lactic acid build up, increase lactic acid clearance, and increase muscle mass.

These and other subjects are effectively treated prophylactically and/or therapeutically by administering to the subject an effective amount of a base solution prepared using a hydroxide or oxide compound such as calcium hydroxide and/or calcium oxide. As noted above, the methods and formulations of the present invention may employ the oxide or hydroxide such as calcium hydroxide and/or calcium oxide in a variety of forms including solvates, hydrates, or combinations thereof in forming the base solution.

In some embodiments, alkaline water may be taken in a concentrated formulation with a pH of between about 12 to about 13.75, preferably about 12 to about 12.5 and a conductivity of about 700 μS/cm to about 2000 μS/cm, preferably about 700 μScm to about 1500 μS/cm, preferably about 700 μS/cm to about 1000 μS/cm, more preferably about 700 μS/cm to about 750 μS/cm. In other embodiments, Alkaline water as described herein may be diluted with water to a pH of about 6.9 to about 7.5, preferably about 6.9 to about 7.2, more preferably about 7.0 and a conductivity 45 μS/cm to about 60 μS/cm, preferably about 50 μS/cm to about 55 μS/cm. Any type of water that will lower the pH may be used for the dilution including, but not limited to, tap, spring, distilled, reverse osmosis, charcoal filtered, mineral-free, or filtered water.

Within additional aspects of the invention, a composition made using the base solution and additional agents may be combinatorially formulated or coordinately administered to yield an effective alkalizing treatment. The compositions and methods of the present invention provide certain advantages in regulating pH in a mammalian subject. The combination of a composition made using calcium hydroxide, calcium oxide or a solvate or hydrate thereof and an additional alkalinity increasing agent will yield an enhanced therapeutic response beyond the therapeutic response elicited by either agent alone.

Useful secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents, herbs and plant extracts, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein.

For example, useful secondary or additional therapeutic agents including alkalinity increasing agents for use within the formulations and methods of the present invention include sodium bicarbonate; a carbonate, a phosphate, or a hydroxide of sodium or potassium; magnesium carbonate; magnesium hydroxide; ammonium carbonate; ammonium bicarbonate; magnesium oxide; sodium or potassium citrate, bicarbonate, sulfate, and benzoate; ascorbate; calcium carbonate; any pharmaceutically acceptable material that causes the pH of an aqueous medium to rise above pH 7.0, or mixtures thereof.

The compositions described herein may additionally contain sweeteners, stabilizers, flavoring, anti-caking agents, flavor protectants, preservatives, anti-foaming agents, colorants, emulsifiers, thickeners and the like.

The compositions described herein may be consumed internally or applied topically. In some embodiments, the compositions described herein may be applied topically in the form of gels, creams, lotions, milks, waxes, water-in-oil or oil-in water emulsions, sprays, suspensions, cleansing products, hair care products and the like.

In some embodiments, the alkalizing treatment may be administered in combination with therapeutic agents other than additional alkalinity increasing agents. Such combinations may increase the effectiveness of therapeutic agents used to treat particular diseases or conditions such as cancer. In further embodiments, such combinations may decrease the required effective amount of the other therapeutic agents. In some embodiments, the alkalizing treatment may facilitate the administration of therapeutic agents or organic substances which are vulnerable to acidic conditions such as those found in the stomach. Additional therapeutic agents that may be administered to treat or prevent acidosis and conditions associated with acidosis include, but are not limited to, probenecid, allopurinol, nonsteroidal anti-inflammatory drugs (NSAIDs), colchicine, corticosteroids, uricosuric agents, xanthine oxidase inhibitors, losartan, fenofibrate, duloxetine, milnacipran, urate oxidase, Y-700, COX-2 inhibitors, analgesics, corticosteroids, disease-modifying anti-rheumatic drugs, antibiotics, vasodepressors, sulfasalazine, radiation therapy, chemotherapy, and benzaldehyde derivatives such as those described in U.S. patent application Ser. No. 12/418,342, incorporated by reference herein in its entirety.

The compositions of the present invention are further effective in preventing secondary infections in mammalian subjects with compromised immune systems, such as those subjects suffering from chronic diseases such as cancer or HIV.

In additional exemplary embodiments, the compositions and methods of the invention employ an OH⁻ composition made from mineral hydroxides and oxides including, but not limited to, calcium hydroxide and/or calcium oxide as an antioxidant and free radical scavenger, providing certain advantages in regulating free radical and other ROS levels in a mammalian subject.

Mammalian subjects amenable for treatment according to the formulations and methods of the invention further include, but are not limited to, human and other mammalian subjects in need of antioxidant treatment or ROS reduction or management, including those suffering from conditions associated with or complicated with excess free radicals such as gout, Lesch-Nyhan syndrome, hemochromatosis. Alzheimer's disease, amyotrophic lateral sclerosis, arthritis, atherosclerosis, cancer, cataracts, chronic obstructive pulmonary disease, diabetes, diabetic neuropathy, coronary artery disease, heart failure, hypertension, inflammatory bowel disease, macular degeneration, multiple sclerosis, Parkinson's disease, fibromyalgia, Reynaud's phenomenon, cellulitis, methicillin-resistant Staphylococcus aureus (MRSA), streptococcus, hepatitis C and reperfusion injury. In addition, the compositions of the present invention have proven effective in the treatment of skin conditions such as psoriasis. Morgellons disease, and fungal infections including but not limited to candidiasis, tinea cruris, and tinea pedis.

These and other subjects are effectively treated prophylactically and/or therapeutically by administering to the subject a free radical reducing effective amount (antioxidant effective amount) of an OH⁻ solution made from a hydroxide or oxide such as calcium hydroxide or calcium oxide as described herein alone or in combination with a secondary antioxidant agent or additional therapeutic agent. As noted above, the methods and formulations of the present invention may employ calcium hydroxide and/or calcium oxide in making the OH⁻ solution and/or an additional antioxidant agent in a variety of forms including solvates, hydrates, or combinations thereof.

The foregoing and other objects, features, aspects and advantages of the present invention will become more apparent from the following detailed description of the invention and examples, which are intended to exemplify non-limiting embodiments of the invention.

### DETAILED DESCRIPTION

Novel methods and compositions have been provided herein for maintaining optimal pH and treating or preventing acidosis, symptoms of acidosis, and conditions caused by or exacerbated by acidosis. The present invention provides a non-corrosive strong base solution (also referred to as an OH⁻ solution, a base solution or Alkaline water) and methods for using the solution for the regulation of physiological pH in vertebrates, including mammals.

The compositions and methods provided herein are suitable for optimizing health and performance; preventing illness; decreasing recovery times from exertion, illness, and injury; increasing energy levels; improving exercise performance; improving hydration; preventing muscle damage after exercise; and increasing stamina during exercise.

Mammalian subjects amenable for treatment according to the formulations and methods of the invention include, but are not limited to, human and other mammalian subjects with acidosis, as well as conditions associated with or complicated by acidosis including gout, abdominal pain, Alzheimer's disease, amyotrophic lateral sclerosis, fungal infections including but not limited to candidiasis, arthritis, atherosclerosis, cancer, cardiovascular disease, cataracts, cellulitis and pancreatic impairment, chronic obstructive pulmonary disease, coronary artery disease, diabetes, diabetic ketoacidosis, ethanol toxicity, exercise fatigue, folliculitis, gout, heart failure, hemochromatosis, hemorrhage, hepatic disease, hepatitis C, hypertension, hyperventilation, hypotension, hypoxia and hypoperfusion, infection, inflammatory bowel disease, irregular heart rhythm, Lesch-Nyhan syndrome, lethargy, macular degeneration, methicillin resistant staphylococcus aureus (MRSA), Morgellons disease, fibromyalgia, multiple sclerosis, nausea, non-Hodgkin's and Burkitt's lymphoma, systemic inflammatory response syndrome (SIRS), Parkinson's disease, psoriasis, regional hypoperfusion, pancreatic impairment, reperfusion injury, respiratory disease, Reynaud's phenomenon, sepsis, severe anemia, shock, tachycardia, tinea cruris, tinea pedis, vomiting and weight gain. In other embodiments, the compositions and methods of the present invention are used as anti-bacterial agents.

Mammalian subjects amenable for treatment according to the formulations and methods of the invention further include, but are not limited to, human and other mammalian subjects with symptoms of acidosis, as well as symptoms or conditions associated with or complicated by acidosis. Such symptoms include, but are not limited to, acidemia, extreme tenderness in the joint, inflammation, swelling, pain, redness in the affected area, confusion, lethargy, rapid breathing, shortness of breath, wheezing, chest pain or pressure, joint stiffness, swelling, joint deformity, crepitus, non-specific fever, joint inflammation, headaches, fatigue, a feeling of euphoria and nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, easy bruising, vomiting, ascites, easy bruising, dry skin, dry mouth, low blood pressure, frequent urination, chest pain, lymph node pain, night sweats, skin rash, hyperventilation, constipation, severe anemia, memory loss, mood swings, musculoskeletal pain and light headedness.

In some embodiments, the compositions and methods of the present invention may be used to speed recovery times, particularly from periods of stress or intense physical activity.

The present invention additionally provides methods of using the non-corrosive strong base solution (also referred to as an OH⁻ solution, a base solution or Alkaline water) as an antioxidant as further described in related U.S. patent application Ser. No. 12/167,123 filed Jul. 2, 2008 which claims priority benefit of U.S. Provisional Patent Application No. 60/967,633 filed Jul. 2, 2007.

The antioxidant compositions described herein may be used in the reduction of reactive oxygen species in vertebrates, including mammals. Reduction of free radicals and other reactive oxygen species (ROS) is effective in the treatment of diseases including, but not limited to, gout, abdominal pain, Alzheimer's disease, amyotrophic lateral sclerosis, arthritis, atherosclerosis, cancer, cardiovascular disease, cataracts, cellulitis and pancreatic impairment, chronic obstructive pulmonary disease, coronary artery disease, diabetes, diabetic ketoacidosis, ethanol toxicity, exercise fatigue, folliculitis, gout, heart failure, hemochromatosis, hemorrhage, hepatic disease, hepatitis C, hypertension, hyperventilation, hypotension, hypoxia, hypoperfusion, infection, inflammatory bowel disease, irregular heart rhythm, Lesch-Nyhan syndrome, lethargy, macular degeneration, methicillin resistant staphylococcus aureus (MRSA), fibromyalgia, multiple sclerosis, nausea, non-Hodgkin's and Burkitt's lymphoma. Parkinson's disease, systemic inflammatory response syndrome (SIRS), psoriasis, regional hypoperfusion, pancreatic impairment, reperfusion injury, respiratory disease. Reynaud's phenomenon, sepsis, severe anemia, shock, tachycardia, vomiting and weight gain.

Another embodiment of the present invention provides methods for treating skin conditions and infections in vertebrates, including mammals, including conditions such as, but not limited to, methicillin-resistant Staphylococcus aureus (MRSA), psoriasis. Morgellons disease and fungal infections including but not limited to candidiasis, tinea cruris, and tinea pedis.

A further embodiment of the present invention provides a strong base solution (also referred to as an OH⁻ solution, a base solution or Alkaline water) for use in the prevention of secondary infections in vertebrates, including mammalian subjects; particularly mammalian subjects with compromised immune systems, such as those subjects suffering from chronic diseases such as, but not limited to, cancer or HIV, or whose immune systems are compromised due to treatments for diseases such as cancer.

An additional embodiment of the present invention provides methods of using the strong base solution to increase the effectiveness of other pharmaceutical agents. The use of the strong base solution neutralizes stomach and other body acids, allowing for increased absorption of certain medications that would otherwise be destroyed or weakened during ingestion.

For the purposes of describing the present invention, the following terms and definitions are provided by way of example. Additional terms and definitions for describing embodiments of the present invention are provided by way of example elsewhere in the application.

As used herein, “microbial” refers to any microorganism capable of causing disease. Such microorganisms include fungal, viral and bacterial microorganisms.

By the term “effective amount” of a compound is meant a non-toxic but sufficient amount of the compound to provide the desired function, i.e., as an anti-infective, as an antioxidant, or as an alkalizing agent. An appropriate effective amount may be determined by one of ordinary skill in the art using only routine experimentation.

Formulations and methods herein employ an OH⁻ solution made from an oxide or hydroxide such as calcium hydroxide or calcium oxide alone or with an additional or secondary therapeutic agent for the regulation of physiological pH. Within these formulations and methods, the secondary agent may be provided in any of a variety of forms, including any polymorphs, enantiomers, pharmaceutically acceptable salts, solvates, hydrates, or combinations thereof. Such combinations of an OH⁻ composition and secondary agent may be administered either combinatorially or coordinately as disclosed herein to effectively treat mammalian subjects with acidosis as well as complications associated with acidosis such as increased infection, cancer, diabetes and pancreatic impairment. Useful secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents, herbs and plant extracts, flavorings, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein. Within these formulations and methods, the secondary agent may be provided in any of a variety of forms, including any polymorphs, enantiomers, pharmaceutically acceptable salts, solvates, hydrates, or combinations thereof. Such combinations of an OH⁻ composition and secondary agent may be administered either combinatorially or coordinately as disclosed herein to effectively treat or prevent acidosis and conditions or symptoms caused by or exacerbated by acidosis.

Formulations and methods herein may additionally employ a base solution as an antioxidant or free radical scavenger for the regulation of ROS levels including free radical levels. Within these formulations and methods, the oxide or hydroxide such as calcium hydroxide or calcium oxide used to produce the OH⁻ solution may be provided in any of a variety of forms, including solvates, hydrates, or combinations thereof. Formulations containing a non-corrosive strong base solution made from calcium hydroxide or calcium oxide as disclosed herein are effectively used to treat mammalian subjects suffering from an over accumulation of free radicals as well as diseases and conditions associated with free radicals including, but not limited to gout. Lesch-Nyhan syndrome, hemochromatosis, Alzheimer's disease, amyotrophic lateral sclerosis, arthritis, atherosclerosis, cancer, cataracts, chronic obstructive pulmonary disease, diabetes, coronary artery disease, heart failure, hypertension, inflammatory bowel disease, macular degeneration, multiple sclerosis. Parkinson's disease. Reynaud's phenomenon, hepatitis C, cellulitis, methicillin-resistant Staphylococcus aureus, reperfusion injury, and skin conditions including, but not limited to, psoriasis, folliculitis, Morgellons disease, candidiasis, tinea cruris, and tinea pedis.

Formulations and methods herein may also employ an OH⁻ composition made from an oxide or hydroxide such as, but not limited to, calcium hydroxide and/or calcium oxide alone or with an additional antioxidant agent as an antioxidant or free radical scavenger. Within the methods and compositions of the invention, a base solution alone or in combination with a second therapeutic agent such as an antioxidant agent or their derivatives are effectively formulated or administered as an antioxidant.

Additional therapeutic agents for use within the compositions of the present invention include antioxidants including, but not limited to, xanthine oxidase inhibitors including, but not limited to allopurinol and folic acid; NADPH oxidase inhibitors, including, but not limited to, adenosine; calcium channel blockers; superoxide dismutases; catalases; albumin; inhibitors of iron redox cycling, including, but not limited to deferoxamine, apotransferin and ceruloplasmin; beta carotene; ascorbates; myricetin-3-O-galactoside, quercitin-3-O-galactoside; and alpha tocopherol.

Formulations and methods for use as an alkalizing agent, antioxidant or ROS scavenger as described herein may additionally employ therapeutic agents such as, but not limited to, probenecid, allopurinol, nonsteroidal anti-inflammatory drugs (NSAIDs), colchicine, corticosteroids, uricosuric agents, xanthine oxidase inhibitors, losartan, fenofibrate, urate oxidase, Y-700, COX-2 inhibitors, analgesics, corticosteroids, disease-modifying anti-rheumatic drugs, antibiotics, vasodepressors, sulfasalazine, radiation therapy, chemotherapy, duloxetin, milnacipran, gabapentin, pregabalin, and benzaldehyde derivatives such as those described in U.S. patent application Ser. No. 12/418,342, incorporated herein by reference in its entirety.

A broad range of mammalian subjects, including human subjects, are amenable for treatment using the formulations and methods of the invention. These subjects include, but are not limited to, human and other mammalian subjects with acidosis and/or excessive free radical production as well as those suffering from conditions or complications of having acidosis including increased susceptibility to microbial infections or other secondary infections; skin infections such as, but not limited to, MRSA; psoriasis; Morgellons disease; and fungal infections such as candidiasis, tinea cruris, and tinea pedis. Additionally amenable to treatment are mammals including humans in need of antioxidant treatment or free radical elimination, including those suffering from conditions or complications associated with excess free radicals, including, but not limited to, gout, Lesch-Nyhan syndrome, hemochromatosis, Alzheimer's disease, amyotrophic lateral sclerosis, arthritis, atherosclerosis, cancer, cataracts, chronic obstructive pulmonary disease, diabetes, cellulitis, coronary artery disease, heart failure, hypertension, inflammatory bowel disease, macular degeneration, multiple sclerosis, Parkinson's disease. Reynaud's phenomenon, hepatitis C, reperfusion injury, MRSA, sepsis, folliculitis, gout, arthritis, hypoxia, hypoperfusion, hemorrhage, ethanol toxicity, shock, hepatic disease, diabetic ketoacidosis, exercise fatigue, non-Hodgkin's and Burkitt's lymphoma, nausea, vomiting, hyperventilation, abdominal pain, lethargy, shock, severe anemia, systemic inflammatory response syndrome (SIRS), hypotension, irregular heart rhythm, tachycardia, weight gain, fibromyalgia, cardiovascular disease, respiratory disease, infection, diabetes, cellulitis and pancreatic impairment and infection.

Mammalian subjects amenable for treatment according to the formulations and methods of the invention further include, but are not limited to, human and other mammalian subjects with symptoms of acidosis, as well as symptoms or conditions associated with or complicated by acidosis. Such symptoms include, but are not limited to, acidemia, extreme tenderness in the joint, inflammation, swelling, pain, redness in the affected area, confusion, lethargy, rapid breathing, shortness of breath, wheezing, chest pain or pressure, joint stiffness, swelling, joint deformity, crepitus, non-specific fever, joint inflammation, headaches, fatigue, a feeling of euphoria and nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, easy bruising, vomiting, ascites, easy bruising, dry skin, dry mouth, low blood pressure, frequent urination, chest pain, lymph node pain, night sweats, skin rash, hyperventilation, constipation, severe anemia, memory loss, mood swings, musculoskeletal pain and light headedness.

Alkaline water, or water with a high concentration of OH⁻ ions as described herein may be manufactured by any means generally used. In some embodiments, alkaline water may be manufactured by combining oxides and hydroxides including, but not limited to, calcium hydroxide, calcium oxide, sodium hydroxide, sodium oxide, potassium hydroxide, potassium oxide, magnesium hydroxide, or magnesium oxide, with water to create a non-corrosive base solution with a high concentration of OH⁻ ions. The water may be tap, spring, mineral-free, filtered, purified, distilled, or any other suitable water with a low mineral concentration.

In some embodiments, the OH⁻ solution of the present invention may be formed through the dissolution of calcium hydroxide in water. In some embodiments, the calcium may be between 2% and 10% mole weight, preferably between 2% and 6% mole weight, more preferably 4% mole weight in water. Dissociation of the calcium hydroxide in water may be facilitated by any means applicable. In some embodiments, the calcium hydroxide solution may be agitated. In other embodiments, the calcium hydroxide solution may be exposed to a magnetic field. In further embodiments, the calcium hydroxide solution may be agitated while being exposed to a magnetic field.

Such manipulation of the solution will yield substantial dissolution of the calcium hydroxide, creating a supersaturated solution. In some embodiments, substantial dissolution is such that the dissociation of the calcium hydroxide is increased to between 50% and 95% of maximum dissociation, preferably between 50% and 75% of maximum dissociation, more preferably between 75% and 95% of maximum dissociation, in some cases greater than 95% dissociation. By maximum dissociation is meant that when additional calcium hydroxide is added to the solution at a given temperature or pressure, the calcium hydroxide precipitates out regardless of the length of time or additional agitation. In some embodiments, agitation of the calcium hydroxide solution in a magnetic field increases the pH of the calcium hydroxide solution to at least one pH unit higher than a normal saturated Ca(OH)₂ solution, preferably 1 to 3 points higher than a normal saturated Ca(OH)₂ solution. In further embodiments, agitating the solution in a strong magnetic field increases the solubility of the Ca(OH)₂ to greater than normal, preferably 2-200 times greater than normal, more preferably 50 to 100 times greater than normal, preferably 100 times greater than normal.

The magnetic field to which the calcium hydroxide solution is exposed may be generated by any means applicable. In some embodiments, the magnetic field may be generated by magnets, magnetic water treatment units or other magnetic field generating apparatus. Such magnetic field generating apparatus may be composed of one or a plurality of magnets which may surround, be placed around, or be otherwise disposed of adjacent to the container containing the Ca(OH)₂ solution. Any kind of magnet or apparatus that creates a strong magnetic field may be used. Magnets which may be used as part of magnetic water treatment units or to otherwise generate a magnetic field include, but are not limited to, NdFeB (Neodymium-Iron-Boron), Ferrite, AlNiCo (Aluminum-Nickel-Cobalt), SmCo (Samarium Cobalt), Alcomax (alloy of iron, nickel, aluminium, cobalt and copper), Cunife (copper, nickel and iron or copper, nickel, iron and cobalt), and Fernico (iron, nickel, cobalt) magnets. The magnets may be monopolar or bipolar. In other embodiments, the magnetic field generating apparatus may comprise electromagnets. In additional embodiments, the magnets may be encased in a housing. Such housing may be made of any material applicable, including, but not limited to, metals such as, but not limited to, aluminum, or steel, and plastics, or any combination thereof. In some embodiments, magnets on opposing sides of the container holding the solution may have opposite poles such that, for example, the positive and negative poles face each other. In other embodiments, the magnets may rotate around the container of calcium hydroxide solution.

In order to increase the OH⁻ concentration of the calcium hydroxide solution, it may be combined with a solution made from sulfuric acid. In order to create the solution made from sulfuric acid, sulfuric acid is added to water. In some embodiments, enough sulfuric acid is added to water to create a solution of equal molar strength to the Ca(OH)₂ in the calcium hydroxide solution. In other embodiments, the concentration of the solution will be about 0.02% to about 0.08% acid in water by volume, preferably about 0.04% to about 0.06% acid in water by volume. In further embodiments the concentration may be about 50-100 mL of sulfuric acid (Baume 12°) per gallon of water, preferably about 70 mL to about 80 mL, more preferably about 70 to about 78 mL of sulfuric acid per gallon of water. In some embodiments, the sulfuric acid solution may be agitated until substantial dissociation occurs such that 75% to 100% of maximum dissociation is achieved, preferably 75% to 95% of maximum dissociation, more preferably 80% to 95% of maximum dissociation of sulfuric acid, in some instances greater than 95% dissociation of sulfuric acid.

In some embodiments, it may be desirable to reduce the acidity of the sulfuric acid solution. The reduction of acidity may occur through any means applicable. In some embodiments, the reduction of acidity may occur through the introduction of additional oxygen to the solution. In one embodiment, nascent oxygen may be introduced into the sulfuric acid solution. In another embodiment, the sulfuric acid solution may be treated with ozone by circulating the solution through ozone generators. The ozone generators dissociate an oxygen which is consumed by (2H+) ion(s) in the acid solution to create water. The acid solution may be re-circulated through the ozone units until a particular concentration of oxygen is absorbed or a particular pH is achieved. In some embodiments, the sulfuric acid solution will be run through the ozone generators until the pH increases by at least 1 to 6 points, preferably at least 1 to 4 points, more preferably at least 2 to 3 points. In some embodiments, the sulfuric acid solution will be circulated through ozone generators until the pH reaches or exceeds about 7.0. The neutralized acid solution may then be slowly added to the calcium hydroxide solution to form a resultant mixture. The free calcium in the calcium hydroxide solution will react with the sulfate ions (SO₄²⁻) in the acid solution to create insoluble anhydrous calcium sulfate precipitate. The mixture may then be agitated until the reaction goes to completion and the anhydrous calcium sulfate may be filtered or otherwise removed from the solution. In some embodiments, a non-ionic surfactant may be added to the resulting mixture in order to enhance precipitation. Such non-ionic surfactants may include, but are not limited to, linear or nonyl-phenol alcohols or fatty acids, alcohol ethoxylates, alkylphenol ethoxylates, alkyl polyglycosides, alkyl ethers such as polyoxyethylene octyl ether, polyoxyethylene lauryl ether, polyoxyethylene stearyl ether, and polyoxyethylene oleyl ether; alkyl phenyl ethers such as polyoxyethylene octylphenyl ether, and polyoxyethylene nonylphenyl ether; alkyl esters such as polyoxyethylene laurate, polyoxyethylene stearate, and polyoxyethylene oleate; alkylamines such as polyoxyethylene laurylamino ether, polyoxyethylene stearylamino ether, polyoxycthylene oleylamino ether, polyoxyethylene soybean amino ether, and polyoxyethylene beef tallow amino ether; alkylamides such as polyoxyethylene lauric amide, polyoxycthylene stearic amide, and polyoxyethylene oleic amide; vegetable oil ethers such as polyoxyethylene castor oil ether, and polyoxyethylene rapeseed oil ether; alkanolamides such as lauric acid diethanolamide, stearic acid diethanolamide, and oleic acid diethanolamide; and sorbitan ester ethers such as polyoxycthylene sorbitan monolaurate, polyoxyethylene sorbitan monopalmitate, polyoxyethylene sorbitan monostearate, and polyoxyethylene sorbitan monooleate.

In some embodiments, calcium oxide (CaO), another strong base, is used to form the acid neutralizing agent and/or antioxidant. Calcium oxide is stirred into purified, distilled, or mineral free water to create a non-corrosive base solution with a high concentration of OH⁻ ions.

In some embodiments, the calcium may be between 2% and 10% mole weight, preferably between 2% and 6% mole weight, more preferably 4% mole weight in water. Dissociation of the calcium oxide in water may be facilitated by any means applicable. In some embodiments, the calcium oxide solution may be agitated.

Such a calcium oxide solution will have a pH between about 7 to about 14, preferably a pH of about 12 to about 14, more preferably about 12.3 to about 13.8, preferably a pH of about 12.5 to about 13.75 and an ionic concentration such that the conductivity would be about 50 μS/cm to about 2000 μS/cm, preferably between about 100 μS/cm to about 1000 μS/cm, preferably about 500 μS/cm to about 800 μS/cm, preferably about 650 μS/cm to about 750 μS/cm, preferably about 700 μS/cm to about 750 μS/cm. In some embodiments, the ionic concentration is such that the conductivity is between 700 μS/cm to about 2000 μS/cm.

The resulting concentrated alkaline water may be consumed directly or diluted with water to a pH of about 7 to about 10, about 7.5 to about 8. The diluted alkaline water may have a conductivity of about 45 μS/cm to about 90 μS/cm, about 50 μS/cm to about 75 μS/cm, about 50 μS/cm to about 60 μS/cm. In some embodiments, the water may be non-chlorinated. In other embodiments, the water may be spring water. In further embodiments, the water may be distilled. In yet another embodiment, the water may be mineral-free water. In additional embodiments, the water may be generated by an alkaline water machine.

In some embodiments, alkaline water may be taken in a concentrated formulation with a pH of between about 12 to about 13.75, preferably about 12 to about 12.5 and a conductivity of about 700 μS/cm to about 2000 μS/cm, preferably about 700 μScm to about 1500 μS/cm, preferably about 700 μS/cm to about 1000 μS/cm, more preferably about 700 μS/cm to about 750 μS/cm. In other embodiments. Alkaline water as described herein may be diluted with purified, distilled, tap, spring, non-chlorinated, or mineral free water to a pH of about 6.9 to about 7.5, preferably about 6.9 to about 7.2, more preferably about 7.0 and a conductivity 45 μS/cm to about 60 μS/cm, preferably about 50 μS/cm to about 55 μS/cm.

In some embodiments, the solution, however formed, may be filtered at various stages to remove particulates. For example, the calcium hydroxide solution may be filtered prior to combining with the sulfuric acid solution and/or the resultant mixture may be filtered to remove particulates. In other embodiments, the resultant mixture may be additionally cooled or partially frozen to create a slurry and further purified, for example through filtration. In one embodiment, the resulting mixture is cooled to below about 36° F. In another embodiment the resulting mixture is cooled to below about 36° F. but above about 35° F.

In some embodiments, the concentrated OH⁻ solution prepared by combining the calcium hydroxide solution and sulfuric acid solution or dissolution of calcium oxide may be diluted with water to reach a specified pH prior to consumption or administration. In some embodiments, the water may be non-chlorinated. In other embodiments, the water may be spring water. In further embodiments, the water may be distilled. In yet another embodiment, the water may be mineral free water. In additional embodiments, the water may be generated by an alkaline water machine. In some embodiments, the resulting mixture may be diluted to a pH of between about 8.0 to about 11, more preferably between about 8.5 to about 9.5, more preferably between 8.5 to about 9.0. This solution may then be used to effectively neutralize acids, to treat acidosis, prophylactically, to reduce free radicals, and/or as an antioxidant.

The acid/alkaline balance in a healthy mammal is generally regulated through the actions of buffers, respiration and renal function. Two forms of acid are generated as a result of normal metabolic processes. Oxidative metabolism produces a large amount of CO₂ daily which is excreted through the lungs. The other form of acid results from the metabolism of dietary protein, resulting in the accumulation at an average rate of approximately 1 mmol per kilogram of body weight, or 50 to 70 mmol per day of acid in an average adult on a typical Western meat containing diet.

The most important mechanism preventing change in the pH of extracellular fluid is the carbonic acid/bicarbonate buffer system. The importance of this buffer pair relates to certain key properties: bicarbonate is present in a relatively high concentration in the extracellular fluid (between 24 and 28 mmol/L) and the components of the buffer system are effectively under physiological control: the CO₂ by the lungs, and the bicarbonate by the kidneys. A shift in pH can be brought about by either a primary change in the bicarbonate concentration (metabolic disturbances) or in the partial pressure of CO₂ in the blood (respiratory disturbances).

Respiratory acidosis results from the accumulation of CO₂ in the body as a result of failure of pulmonary ventilation. This may occur from lesions either in the central nervous system (e.g. depression of cerebral function, spinal cord injury), in the peripheral nervous pathways involved in ventilating the lungs (peripheral nerve and muscle disorders), in some forms of lung disease involving impaired gas diffusion (e.g. emphysema, asthma, bronchitis, pneumonia, lung cancer or aspiration), or due to pharmaceutical causes.

Metabolic acidosis may result from inorganic acid addition, i.e. the infusion or ingestion of HCl or NH₄Cl; or through gastrointestinal base loss through conditions such as diarrhea, small bowel fistula/drainage, surgical diversion, and renal tubular disorders; stimulation of chemoreceptors; lactic acid accumulation; poison; or diet. The OH⁻ solution of the present invention is effective in the treatment of acidosis regardless of cause.

Alkalinity increasing compositions of the invention typically comprise an amount of a base solution made from calcium hydroxide and/or calcium oxide or another elemental oxide or hydroxide, its solvates, hydrates, or combinations thereof, which is effective for the treatment or prevention of acidosis, as well as complications and related conditions thereof in a mammalian subject. Alkaline water may be taken alone, or in a coordinate or combined formulation with one or more additional agents to optimize health and performance; prevent illness; decrease recovery times from exertion, illness and injury; and extend endurance during exercise. Useful secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents, herbs and plant extracts, flavorings, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein. Within these formulations and methods, the secondary agent may be provided in any of a variety of forms, including any polymorphs, enantiomers, pharmaceutically acceptable salts, solvates, hydrates, or combinations thereof.

Typically, an alkalinity increasing effective amount of an OH⁻ formulation will comprise an amount of the active compound which is therapeutically effective by itself or with one or more secondary agents, in a single or multiple unit dosage form, taken or applied over a specified period of therapeutic intervention, to measurably alleviate one or more symptoms of acidosis or related conditions in the subject. Within exemplary embodiments, these compositions are effective within in vivo treatment methods to alleviate acidosis. The compositions described herein may additionally contain sweeteners, stabilizers, flavoring, anti-caking agents, flavor protectants, preservatives, fragrances, anti-foaming agents, colorants, emulsifiers and the like. The active compound may be optionally formulated with a pharmaceutically acceptable carrier and/or various excipients, vehicles, stabilizers, buffers, preservatives, fragrances, thickeners, etc.

In addition to generating CO₂, oxidative metabolism may also cause oxidative stress. Oxidative stress is imposed on cells as a result of an increase in oxidant generation (including reactive oxygen species), a decrease in antioxidant protection, or a failure to repair oxidative damage. It has been shown to lower intracellular pH (Mulkey, 2004). It is believed that intracellular and extracellular advanced glycation (AGEs) or lipoxidation end products (ALEs), together with dysregulated glucose and lipid metabolism, are important contributors to oxidant stress, enhanced cellular redox-sensitive transcription factor activity, and impaired innate immune defense, causing inappropriate inflammatory responses mediated in part by reactive oxygen species.

Oxygen has two unpaired electrons in separate orbitals in its outer shell. Sequential reduction of molecular oxygen leads to the formation of a group of reactive oxygen species including the superoxide anion, peroxide and hydroxyl radicals. Oxygen-derived radicals are generated constantly as part of normal aerobic life as oxygen is reduced along the electron transport chain in mitochondria. Reactive oxygen species are also formed as necessary intermediates in a variety of enzyme reactions.

However, these highly reactive radicals can also start a chain reaction which disrupts cellular function. While they are a natural byproduct of metabolic function as well as part of phagocytosis, an excess of free radicals can occur for a variety of reasons. For example, an increase in the production of free radicals can be produced by drugs such as antibiotics that depend on quinoid groups or bound metals for activity (nitrofurantoin), antineoplastic agents as bleomycin, anthracyclines (adriamycin) and methotrexate. In addition, radicals derived from penicillamine, phenylbutazone, some fenamic acids and the aminosalicylate component of sulphasalazine are currently believed to inactivate protease and deplete ascorbic acid accelerating lipid peroxidation. Free radical production may also be increased by radiation, smoking, and inhalation of inorganic particles also known as mineral dust (e.g. asbestos, quartz, and silica). Fever, excess glucocorticoid therapy and hyperthyroidism also increase the generation of oxygen-derived radicals due to increased metabolism. Furthermore, a wide variety of environmental agents including photochemical air pollutants such as pesticides, solvents, anesthetics, exhaust fumes and aromatic hydrocarbons can cause free radical damage to cells.

Free radical and ROS damage can be inhibited by antioxidants. An antioxidant is a substance that when present in low concentrations relative to the oxidizable substrate significantly delays or reduces oxidation of the substrate. Antioxidants protect the body by reacting with free radicals and other reactive oxygen species within the body, hindering oxidation and reducing the amount of circulating free radicals. However, antioxidant supply is limited as an antioxidant molecule can only react with a single free radical. Therefore, there is a constant need to replenish antioxidant resources, whether endogenously or through supplementation. The compositions and methods of the present invention are effective as antioxidants for the elimination and/or reduction of reactive oxygen species including free radicals, regardless of the source of the free radicals.

Antioxidant compositions of the invention typically comprise an amount of a base solution made from calcium hydroxide or calcium oxide, or other elemental oxides and hydroxides, its solvates, hydrates, or combinations thereof, which is effective for the treatment or prevention of excess free radicals as well as complications and related conditions thereof in a mammalian subject. Typically, an antioxidant effective amount (or free radical reducing effective amount) of an OH⁻ formulation of the present invention will comprise an amount of the active compound which is therapeutically effective, in a single or multiple unit dosage form, over a specified period of therapeutic intervention, to measurably alleviate one or more symptoms of free radical damage or related conditions in the subject. The active compound may be optionally formulated with a pharmaceutically acceptable carrier and/or various excipients, vehicles, stabilizers, buffers, preservatives, etc.

The amount, timing and mode of delivery of compositions of the invention comprising an effective amount of a base solution either as an alkalinity increasing agent, (antioxidant agent, free radical reducing agent) will be routinely adjusted on an individual basis, depending on such factors as weight, age, gender, and condition of the individual, the severity of the acidosis and/or free radical damage or related symptoms, whether the administration is prophylactic or therapeutic, and on the basis of other factors known to effect drug delivery, absorption, pharmacokinetics, including, but not limited to, half-life, and efficacy.

An effective dose or multi-dose treatment regimen for the instant alkalinity increasing or antioxidant formulations will ordinarily be selected to approximate a minimal dosing regimen that is necessary and sufficient to substantially prevent or alleviate acidosis or excess free radicals and related conditions in the subject. A dosage and administration protocol will often include repeated dosing therapy over a course of several days or even one or more weeks, months, or years. An effective treatment regime may also involve prophylactic dosage administered on a day or multi-dose per day basis lasting over the course of days, weeks, months or even years.

An “effective amount,” “therapeutic amount,” “therapeutic effective amount,” or “effective dose” is an amount or dose sufficient to elicit a desired pharmacological or therapeutic effect in a mammalian subject; typically resulting in a measurable increase in alkalinity or reduction in free radicals.

Therapeutic efficacy can alternatively be demonstrated by a measurement of blood gases, electron spin resonance, spin trapping, fingerprinting, measurement of free radical markers, liquid chromatography, measurement of markers of oxidative stress, or by altering the nature, recurrence, or duration of conditions associated with acidosis and/or excess free radicals including, but not limited to, Lesch-Nyhan syndrome, hemochromatosis, Alzheimer's disease, amyotrophic lateral sclerosis, atherosclerosis, cataracts, chronic obstructive pulmonary disease, coronary artery disease, heart failure, hypertension, inflammatory bowel disease, macular degeneration, multiple sclerosis, Parkinson's disease. Reynaud's phenomenon, reperfusion injury, pancreatic impairment, methicillin-resistant Staphylococcus aureus (MRSA), hepatitis C, cellulitis, sepsis, folliculitis, fibromyalgia, gout, arthritis, hypoxia and hypoperfusion, hemorrhage, ethanol toxicity, hepatic disease, diabetic ketoacidosis, exercise fatigue, systemic inflammatory response syndrome (SIRS), regional hypoperfusion, non-Hodgkin's and Burkitt's lymphoma, nausea, vomiting, hyperventilation, abdominal pain, lethargy, shock, severe anemia, hypotension, irregular heart rhythm, tachycardia, weight gain, cancer, cardiovascular disease, respiratory disease, infection, diabetes, cellulitis and pancreatic impairment and infection. Therapeutic effectiveness may further be demonstrated by a reduction in the symptoms of skin conditions such as psoriasis, MRSA, Morgellons disease and fungal infections such as candidiasis, tinea cruris, and tinea pedis. Therapeutic effectiveness may additionally be demonstrated by a reduction in the number of secondary infections experienced by a subject, particularly in a subject with a compromised immune system.

Therapeutic effectiveness may further be demonstrated by a decrease in the symptoms of the conditions being treated, for instance, a decrease in acidemia, hyperlactemia, lactic acidosis, lactic acid build up, abscesses, boils, redness, pain, headache, a general sick feeling, muscle aches, shortness of breath, fatigue, fever, shivering and chest pain of mild to medium intensity, muscle aches, joint pain, bone pain, chest pain, painful breathing, shortness of breath, fever and chills, low blood pressure, fatigue, headaches, rash, malaise, septic shock, septic arthritis, abscesses deep within the body, blood poisoning, or septicemia, a bone infection called osteomyelitis, meningitis, endocarditis, pneumonia, joint inflammation, confusion, lethargy, rapid breathing, shortness of breath, wheezing, chest pain or pressure, joint stiffness, swelling, joint deformity, crepitus, non-specific fever, joint inflammation, headaches, fatigue, a feeling of euphoria, nausea, seizures, coma, generalized weakness, abnormal heart function, decreased platelet count, areas of mottled skin, fever, low blood pressure, tachycardia, skin discoloration, irregular heartbeat, loss of appetite, jaundice, abdominal pain, memory loss, mood swings, musculoskeletal pain, easy bruising, nausea, vomiting, ascites, easy bruising, dry skin, dry mouth, low blood pressure, frequent urination, chest pain, lymph node pain, night sweats, skin rash, hyperventilation, constipation, severe anemia, and light headedness.

Therapeutic effectiveness may also be demonstrated by a decrease in the amount of other pharmaceutical agents necessary to treat a disease, or an increase in the effectiveness of current dosages. For example, the compositions of the present invention may increase the effectiveness of chemotherapeutic agents, decreasing the amount of chemotherapeutic agents needed or the length of the treatment needed.

Therapeutic effectiveness may be determined, for example, through an arterial blood gas. In an arterial blood gas test, arterial blood is taken from any easily accessible artery (typically either radial, brachial, or femoral) or out of an arterial line. Once the sample is obtained, care should be taken to eliminate visible gas bubbles, as these bubbles can dissolve into the sample and cause inaccurate results. The sealed syringe is then taken to a blood gas monitor. The machine aspirates the blood from the syringe and measures the pH and the partial pressures of oxygen and carbon dioxide and the bicarbonate concentration, as well as the oxygen saturation of hemoglobin. Normal pH of blood is between about 7.4 and 7.3, preferably 7.365, Effective amounts of the mixtures of the present invention will increase plasma pH from below 7.0 to a pH of about 7.6 to 7.3. Effective alkalinity increasing amounts may increase plasma pH of 6.0 to a pH of about 6.5, preferably to about 6.7, more preferably to about pH 7.0, preferably to a pH of 7.4 or higher. Effective alkalinity increasing amounts may increase plasma pH of 6.0 to a pH of about 6.5, preferably to about 6.7, more preferably to about pH 7.0, preferably to a pH of 7.4 or higher.

Therapeutic effectiveness may also be demonstrated through a litmus test in which a sample of saliva is taken upon awakening and tested with a strip of litmus paper. A urine sample may also be tested with a strip of litmus paper or a litmus test strip. The litmus paper is then compared to a litmus scale to determine the pH of the sample. Optimally, the pH of saliva is about 7.4 and the pH of urine is about 6.6. The methods and compositions of the present invention are therapeutically effective to increase the pH of saliva and/or urine by about 2-40%, 5-15%, 10-20% or more.

Therapeutic effectiveness may additionally be determined using a Lactic acid meter. During intense exercise, physiological pH levels increase indicating an increase in acid in the body. The effect of alkaline water on lowering elevated physiological pH can be determined using a lactic acid meter. Measurements may be taken before, during, and after intense activity. An effective amount of an Alkaline water composition would maintain normal or decrease elevated levels of physiological pH during exercise. In some embodiments, alkaline water consumed during exercise will decrease the drop in physiological pH. In other embodiments, Alkaline water consumed during exercise will prevent a drop in physiological pH. In additional embodiments, alkaline water consumed after exercise will increase the rate at which physiological pH returns to baseline levels. In some embodiments, the consumption of Alkaline water as described herein during exercise will increase an individual's maximal lactate steady state allowing them to exercise longer and harder than had previously been possible.

Therapeutic effectiveness as a free radical scavenger may further be demonstrated through electron spin resonance. Electron spin resonance (ESR) is a spectroscopic technique which detects species that have unpaired electrons such as free radicals. The degeneracy of the electron spin states characterized by the quantum number, m_{S}=±½, is lifted by the application of a magnetic field and transitions between the spin levels are induced by radiation of the appropriate frequency. An unpaired electron interacts with its environment, and the details of ESR spectra depend on the nature of those interactions. The integrated intensity of the spectrum is proportional to the concentration of radicals in the sample. An effective free radical reducing or antioxidant amount of the mixture of the present invention will decrease the intensity of the spectrum by 2-50%, 10-40%, 15-30%, 20-25% or more.

Spin trapping provides a nitrone or nitrose compound for an addition reaction which produces an electron spin resonance spectroscopy-detectable aminoxyl radical. The product of the reaction can then be measured through electronic resonance spectroscopy. Effectiveness of the compositions of the present invention as a free radical scavenger may be demonstrated by a decrease in the product of the reaction by 2-50%, 1040%, 15-30%, 20-25% or more.

Oxidative stress as a result of free radical production can be measured in myriad ways including, microplate cold light cytofluorimetry, and measurement of coiling of DNA, and cytochrome C production. Oxidative stress caused by free radicals may also be determined through measurement of thiobarbituric acid reacting substances, measurement of pentane and ethane, measurement of creatine kinase, and measurement of conjugated dienes. Effective free radical reducing or antioxidant amounts of the composition of the present invention will reduce the amount of the measured markers by 2-50%, 10-40%, 15-30%, 20-25% or more.

In microplate cold light cytofluroimetry, permeable probes are inserted directly in living cells using a method of UV and visible fluorescent detection. Some particles are known to have fluorescence (such as cigarette smoke particles) when they react with free radicals and the amount of free radical damage can be assessed by the amount of fluorescence in a sample. A quantitative measure can be obtained using a flow cytometer. This method also evaluates intracellular glutathione and hydrogen peroxide production. A free radical reducing effective amount of a mixture of the present invention will decrease the fluorescence by 2-50%, 10-40%, 15-30%, 20-25% or more.

Therapeutic effectiveness of the solution as a free radical scavenger may further be demonstrated by the measurement of the proportion a relaxed coil DNA to that of supercoiled DNA wherein X=Relaxed coil DNA/Supercoiled DNA. Plasmid DNA is incubated with 5 μl of particle suspensions at 37° C. in a water bath. The supercoiled, relaxed coiled and linearised plasmid DNA are separated by electrophoresis and quantified by scanning. The higher the value of X, the more oxidative damage (based on free radicals damaging the supercoiled DNA and causing it to uncoil). An effective free radical reducing or antioxidant amount of a mixture of the present invention will decrease the value of X by 2-50%, 10-40%, 15-30%, 20-25% or more.

The rate of Cytochrome C reduction can be measured using luminol induced chemiluminescence for quantifying the results. Therapeutically effective free radical reducing or antioxidant amounts of the solution of the present invention will decrease the rate of cytochrome C reduction by 2-50%, 10-40%, 15-30%, 20-25% or more.

Therapeutic effectiveness may be demonstrated by a decrease or absence of an infection. For example, MRSA may be detected by culture, blood test, skin culture from the infected site, culture of the drainage from the infection, urine culture, and sputum culture. An effective treatment with the OH⁻ composition according to the formulations and methods of the invention will decrease MRSA levels by 5%. 10%, 20%, 30%, 50% or greater reduction, up to a 75-90%, or 95% or greater, reduction.

Following administration of the OH⁻ composition according to the formulations and methods of the invention, test subjects will exhibit a 5%, 10%, 20%, 30%, 50% or greater reduction, up to a 75-90%, or 95% or greater, reduction, in one or more symptoms associated with acidosis or excessive free radical production as compared to placebo-treated or other suitable control subjects. Test subjects may also exhibit a 10%, 20%, 30%, 50% or greater reduction, up to a 75-90%, or 95% or greater, reduction, in the symptoms of one or more conditions associated with acidosis or excessive free radical production including, but not limited to, gout, abdominal pain, Alzheimer's disease, amyotrophic lateral sclerosis, fibromyalgia, fungal infections including but not limited to candidiasis, arthritis, atherosclerosis, cancer, cardiovascular disease, cataracts, cellulitis and pancreatic impairment, chronic obstructive pulmonary disease, coronary artery disease, diabetes, diabetic ketoacidosis, ethanol toxicity, exercise fatigue, folliculitis, gout, heart failure, hemochromatosis, hemorrhage, hepatic disease, hepatitis C, hypertension, hyperventilation, hypotension, hypoxia and hypoperfusion, infection, inflammatory bowel disease, irregular heart rhythm, Lesch-Nyhan syndrome, lethargy, macular degeneration, methicillin resistant staphylococcus aureus (MRSA), Morgellons disease, multiple sclerosis, nausea, non-Hodgkin's and Burkitt's lymphoma, Parkinson's disease, psoriasis, regional hypoperfusion, reperfusion injury, respiratory disease, systemic inflammatory response syndrome (SIRS), Reynaud's phenomenon, sepsis, severe anemia, shock, tachycardia, tinea cruris, vomiting and weight gain.

The pharmaceutical compositions of the present invention may be administered by any means that achieves the intended therapeutic or prophylactic purpose. Suitable routes of administration for alkalizing and antioxidant compositions of the invention comprising OH⁻ solutions include, but are not limited to, oral, buccal, nasal, aerosol, mucosal, injectable, slow release, controlled release, iontophoresis, sonophoresis, and other conventional delivery routes, devices and methods. Injectable delivery methods are also contemplated, including but not limited to, intravenous, intramuscular, intraperitoneal, intraspinal, intrathecal, intracerebroventricular, intraarterial, and subcutaneous injection.

Within additional aspects of the invention, combinatorial formulations and coordinate administration methods are provided which employ an effective amount of OH⁻ compositions, and one or more additional active agent(s) that is/are combinatorially formulated or coordinately administered with the OH⁻ solution—yielding an effective formulation or method to modulate, alleviate, treat or prevent acidosis or excessive free radicals in a mammalian subject. Exemplary combinatorial formulations and coordinate treatment methods in this context employ a base solution in combination with one or more additional or adjunctive therapeutic agents.

Such secondary or additional agents for use within the formulations and methods of the present invention include, but are not limited to, alkalinity increasing agents, adaptogens, amino acids and amino acid derivatives, anti-inflammatory agents, anti-nausea agents, analgesics, antioxidants, aphrodisiacs, detoxifying agents, dietary supplements, herbal supplements, calming agents, herbs and plant extracts, flavorings, essential nutrients, coenzymes, electrolytes, energy boosters, essential trace elements, flavonoids, hormones, immune boosters, neurotransmitters, essential fatty acids, memory enhancers, vitamins and minerals, protein, sedatives, stimulants and nutritional supplements for use within the formulations and methods described herein. Within these formulations and methods, the secondary agent may be provided in any of a variety of forms, including any polymorphs, enantiomers, pharmaceutically acceptable salts, solvates, hydrates, or combinations thereof. Such combinations of an OH⁻ composition and secondary agent may be administered either combinatorially or coordinately as disclosed herein to effectively optimize health and performance; prevent illness; decrease recovery times from exertion, illness, and injury; increase energy levels; improve exercise performance; improve hydration; prevent muscle damage after exercise; and increase stamina during exercise.

Such additional or adjunctive therapeutic agents may be additional alkalinity increasing agents including, but not limited to sodium bicarbonate; a carbonate, a phosphate, or a hydroxide of sodium or potassium; magnesium carbonate; magnesium hydroxide; ammonium carbonate; ammonium bicarbonate; magnesium oxide; sodium or potassium citrate, bicarbonate, sulfate, and benzoate; ascorbate; calcium carbonate; or any pharmaceutically acceptable material that causes the pH of an aqueous medium to rise above pH 7.0, or mixtures thereof.

Additional or adjunctive therapeutic agents may also include antioxidants including, but not limited to, xanthine oxidase inhibitors, including, but not limited to, allopurinol and folic acid; NADPH oxidase inhibitors, including, but not limited to, adenosine; calcium channel blockers; superoxide dismutases; catalases; albumin; inhibitors of iron redox cycling, including, but not limited to deferoxamine, apotransferin and ceruloplasmin; beta carotene; ascorbates; myricetin-3-O-galactoside, quercitin-3-O-galactoside; alpha tocopherol; and benzaldehyde derivatives, such as those described in U.S. patent application Ser. No. 12/418,342, incorporated by reference herein in its entirety. Further additional or adjunctive therapeutic agents may include but are not limited to, probenecid, allopurinol nonsteroidal anti-inflammatory drugs (NSAIDs), colchicine, corticosteroids, uricosuric agents, xanthine oxidase inhibitors, losartan, fenofibrate, urate oxidase, Y-700, COX-2 inhibitors, analgesics, corticosteroids, disease-modifying anti-rheumatic drugs, antibiotics, vasodepressors, sulfasalazine, radiation therapy, chemotherapy, duloxetin, milnacipran, gabapentin, pregabalin, and benzaldehyde derivatives such as those described in U.S. patent application Ser. No. 12/418,342, incorporated herein by reference in its entirety.

Adaptogen agents for use within the formulations and methods herein include, but are not limited to, ashwagandha, eleutherococcus senticosus, reishi, astragalus, licorice root, panax quinquefolius, panax ginseng and schisandra berries.

Antioxidants included in the formulations provided herein may be in the form of nutritional supplements such as, but not limited to, vitamin A; vitamin C; vitamin E; erythorbic acid; beta-carotene; carotenes; lutein; manganese; lycopene; melatonin; or coenzyme Q10; xanthine oxidase inhibitors, including, but not limited to, allopurinol and folic acid; NADPH oxidase inhibitors, including, but not limited to, adenosine; calcium channel blockers; superoxide dismutases; catalases; albumin; inhibitors of iron redox cycling, including, but not limited to deferoxamine, apotransferin and ceruloplasmin; beta carotene; ascorbates; myricetin-3-O-galactoside, quercitin-3-O-galactoside; alpha tocopherol; and benzaldehyde derivatives, such as those described in U.S. patent application Ser. No. 12/418,342, incorporated by reference herein in its entirety. In some embodiments, antioxidants may be present in plant extracts which may also be combined with the alkaline water. Plant extracts may come from plant sources such as, but not limited to, apricot, acai fruit, acerola, apple, blueberry, blackberry, black currant, carrots, cherry, chokeberry, cranberry, elderberry, green tea, goji berry, grape seed, mangosteen, maqui berry, milk thistle, pomegranate seed, prune, raspberry, red grape, rooibos, rosehips, strawberry, seabuckthorn, white grape, whole grape, yumberry and acerola fruit.

Vitamin, mineral and nutritional supplements for use herein may be in a variety of forms including, but not limited to, vitamin B complex, folic acid, niacin, niacinamide, pantothenic acid, pyridoxine HCl, vitamin B2, folate, biotin, vitamin C, vitamin D, vitamin D₃, vitamin E, vitamin K, cyanocobalamin, inositol, thiamine, thiamine mononitrate, calcium pantothenate, mixed tocophyerols, d-alpha tocopheryl acetate, magnesium, calcium, calcium carbonate, calcium chelate, calcium di-phosphate, calcium phosphate, iron, magnesium carbonate, magnesium citrate, magnesium oxide, magnesium phosphate, manganese chelate, manganese sulfate, potassium, potassium chelate, potassium chloride, sodium, zinc, vanadyl sulphate, chromium, chromium chloride, chromium picolinate, and chromium polynicotinate.

Amino acids, amino acid precursors and derivatives as used within the formulations herein may be branched or straight chain amino acids. Exemplary amino acids, precursors and derivatives which may be used in the formulations and methods described herein include, but are not limited to, 5-HTP, arginine, beta alanine, carnitine fumarate, citrulline malate, glutamine peptide, glycine, l-alanine, l-arginine, l-arginine hydrochloride, l-histidine, l-methionine, l-lysine HCl, l-phenylalanine, leucine ethyl ester, l-glutamine, l-isoleucine, l-theanine, l-tyrosine, phenylalanine, taurine, tri-methyl glycine, tryptophan, tyrosine, l-carnitine, l-carnosine, glutamine alpha ketoglutarate and alpha-L-polylactate.

Electrolytes used with the formulations herein include, but are not limited to, sodium chloride, sodium acetate, acidic sodium citrate, acidic sodium phosphate, sodium chloride, sodium bicarbonate, sodium bromide, sodium citrate, sodium lactate, sodium molybdate, sodium phosphate, anhydrous sodium sulphate, sodium sulphate, sodium tartrate, sodium benzoate, sodium selenite, and other sodium salts and mixtures thereof; potassium chloride, potassium acetate, potassium bicarbonate, potassium bromide, potassium citrate, potassium-D-gluconate, monobasic potassium phosphate, potassium tartrate, potassium sorbate, potassium iodide, and other potassium salts and mixtures thereof; magnesium carbonate, magnesium citrate, magnesium oxide, magnesium phosphate, as well as other magnesium salts and mixtures thereof; calcium chloride, calcium carbonate, calcium chelate, calcium di-phosphate, calcium lactate, calcium phosphate tribasic and other calcium salts and mixtures thereof. Such electrolytes may be included in the formulations described herein in proportions and amounts suitable to replenish salts lost during exercise or illness or otherwise depleted.

Anti-inflammatory agents for use within the formulations and methods herein include, but are not limited to, extracts from plants such as maqui berry, milk thistle, skull cap, red raspberry, red sour cherry, green tea and hops.

Other agents which may be used in the compositions and methods described herein include anti-nausea agents including, but not limited to, extracts from peppermint, ginger and chamomile.

A further agent which may be used in the compositions and methods described herein includes analgesic agents such as, but not limited to, white willow bark.

The formulations and methods described herein may additionally include herbal supplements and extracts with beneficial properties including, but not limited to, passion flower, horny goat weed, skullcap, milk thistle, Echinacea, dandelion leaf, St. John's wort, green tea, black tea, chamomile or peppermint, or an extract thereof.

The formulations and methods described herein may further include plants with beneficial properties including, but not limited to, guarana seeds, acerola berries, coconut water, yerba mate, acai berry, ginseng root, panax ginseng root, ginkgo biloba, white willow bark, acacia, ashwagandha, chokeberry, elderberry, cranberry, maqui berry, blueberry, pomegranate, rooibos, goji berry, elder berry, valerian, seabuckthorn, yumberry, blackberry, astragalus, damiana, and ginger.

Energy boosters that may increase performance and are contemplated for use within the methods and formulations described herein include, but are not limited to, creatine ethyl ester, creatine monohydrate, magnesium creatine chelate, creatine hydrochloride, creatine nitrate, creatine monohydrate and royal jelly.

Useful flavonoids within the compositions and methods of the present invention are present in chamomile extract, cocoa powder, red grape, black tea, and white tea, ginkgo biloba, berries, parsley, and green tea some or all of which may be included in the compositions and methods described herein.

Useful sedatives for use within the compositions and methods described herein include, but are not limited to, lavender, lemon balm, lemongrass, linden, oatstraw, St. John's wart, valerian root, kava kava, hops and passion flower.

Stimulants for use within the methods and compositions described herein include, but are not limited to, caffeine, citicoline, d-glucuronolactone, guarana extract, ginseng, concentrated green tea, green coffee beans, glucuronolactone, guarana, panax ginseng, panax quinquefolius, Siberian ginseng, and theobromine.

Additional agents which may be included in the formulations and methods described herein are immune boosters including, but not limited to, Echinacea and astragalus root.

Flavoring agents for use with the compositions and methods described herein include, but are not limited to fruit juice, vegetable juice, milk solids, fruit flavors, herbal flavor and mixtures thereof. The fruit juice can be any citrus juice, non-citrus juice, or mixture thereof, which is known for use in dilute juice beverages. The juice can be derived from, but not limited to, apple, cranberry, pear, peach, plum, apricot, nectarine, grape, guava, cherry, currant, raspberry, gooseberry, elderberry, blackberry, blueberry, strawberry, lemon, lime, mandarin, orange, tomato, lettuce, dandelion, rhubarb, pineapple, coconut, pomegranate, kiwi, mango, papaya, banana, watermelon, passion fruit, tangerine, and cantaloupe. The vegetable juice can be any vegetable juice generally consumed including but not limited to, celery, spinach, cabbage, watercress, carrot, beet, spirulina, sweet potato, kale, romaine, collard greens, endive, escarole, bok choy, fennel, parsley, wheat grass, or cucumber. Such fruit and vegetable juices may or may not have additional beneficial properties such as antioxidants and/or flavonoids.

Formulations and methods herein may additionally include a protein source. Protein sources include, but are not limited to, milk solids, calcium caseinate, whey protein concentrate, whey protein isolate, whey protein hydrolysate, soy protein, casein hydrolysate, rice protein, wheat protein, corn protein, partially hydrolyzed whey protein, or ultra-filtered whey protein.

Formulations and methods herein may further include one or more sweeteners or other carbohydrate source. Such sweeteners include, but are not limited to, acesulfame potassium, aspartame, cane sugar, corn syrup, crystalline fructose, dextrose, D-ribose, fructose, glucose, glucose-fructose syrup, high fructose corn syrup, high fructose liquid sugar, honey, maltodextrin, sorbitol, stevia, sucralose, sucrose, sugar, trehalose, truvia or xylitol.

The use of these additional or adjunctive therapeutic agents in conjunction with the alkalizing or antioxidant agent of the present invention may increase the effectiveness of the therapeutic agents and/or decrease the amount of such agents that may be required.

In some embodiments, the alkalinity increasing agent may be administered in conjunction with an additional therapeutic agent to facilitate consumption of the additional therapeutic agent. For example, some therapeutic agents may be extremely acidic. Such agents may be administered in conjunction with the alkalinity increasing agent to neutralize the acidity and increase the forms of administration that would be acceptable. In another embodiment, the alkalinity increasing agent may be used to temporarily neutralize stomach acid or other acid conditions so that therapeutic agents which are destroyed by acid such as, but not limited to, nutritional supplements or other organics such as vitamins, including vitamin B₁₂, can be ingested.

In certain embodiments, the invention provides combinatorial alkalizing or antioxidant formulations comprising a base solution made from calcium hydroxide and/or calcium oxide and one or more adjunctive agent(s) having alkalizing or antioxidant activity, or both, or additional adjunctive agents which may have neither alkalizing nor antioxidant activity but which are useful in the treatment of underlying conditions or prophylactically. Within such combinatorial formulations, the OH⁻ solution and the adjunctive agent(s) having alkalizing and/or antioxidant activity, or non-alkalizing/antioxidant agents will be present in a combined formulation in effective amounts, alone or in combination. In exemplary embodiments, a base solution and a non-calcium hydroxide based alkalizing and/or antioxidant agent will each be present in an alkalizing and/or antioxidant amount (i.e., in singular dosage which will alone elicit a detectable alkalizing or free radical reduced response in the subject). Alternatively, the combinatorial formulation may comprise one or both of the OH⁻ solution and a non-calcium hydroxide based alkalizing and/or antioxidant agent or other adjunctive agent in sub-therapeutic singular dosage amount(s), wherein the combinatorial formulation comprising both agents features a combined dosage of both agents that is collectively effective. Effectiveness may elicit an alkalizing or free radical reducing response or other increased therapeutic response. Thus, one or both of the OH⁻ solution and a non-calcium hydroxide based alkalizing and/or antioxidant agents may be present in the formulation, or administered in a coordinate administration protocol, at a sub-therapeutic dose, but collectively in the formulation or method they elicit a detectable alkalizing and/or antioxidant response in the subject.

To practice the coordinate administration methods of the invention, an OH⁻ mixture is administered, simultaneously or sequentially, in a coordinate treatment protocol with one or more of the secondary or adjunctive therapeutic agents contemplated herein. The coordinate administration may be done simultaneously or sequentially in either order, and there may be a time period while only one or both (or all) active therapeutic agents, individually and/or collectively, exert their biological activities. A distinguishing aspect of all such coordinate treatment methods is that the OH⁻ solution exerts at least some detectable alkalizing or antioxidant activity, and/or elicits a favorable clinical response, which may or may not be in conjunction with a secondary clinical response provided by the secondary therapeutic agent. Often the coordinate administration of a base solution with a secondary therapeutic agent as contemplated herein will yield an enhanced therapeutic response beyond the therapeutic response elicited by either or both the OH⁻ solution and/or secondary therapeutic agent alone.

The amount, timing and mode of delivery of compositions of the invention comprising an effective amount of a base solution of the present invention will be routinely adjusted on an individual basis, depending on such factors as weight, age, gender, and condition of the individual, the severity of the acidosis, ROS levels including free radical production or related symptoms, whether the administration is prophylactic or therapeutic, and on the basis of other factors known to effect drug delivery, absorption, pharmacokinetics, including, but not limited to, half-life, and efficacy.

Effective doses may be extrapolated from dose-response curves derived from in vitro or animal model test systems. Such animal models and systems are well known in the art. The precise dose to be employed will also depend on the route of administration, the seriousness of the disease or disorder, and body size, and should be decided according to the judgment of the practitioner and each patient's circumstances. However, suitable dosage ranges for oral administration are generally about 5 ounces (0.147 L) to about 135.256 ounces (4 L) of the diluted OH⁻ solution (having a pH between 7.5 and 9.5) per day. In specific preferred embodiments of the invention, the oral dose is about 5 ounces (0.147 L) to about 100 ounces (2.9 L), about 5 ounces (0.147 L) to about 90 ounces (2.6 L) of OH⁻ solution per day, more preferably about 8 ounces (0.236 L) to about 80 ounces (2.36 L) of OH⁻ solution per day, more preferably about 24 ounces (0.71 L) to about 32 ounces (0.94 L) per day, more preferably about 32 ounces (0.94 L) to about 48 ounces (1.4 L) per day, more preferably about 35 ounces (1.035 L) to about 80 (2.36 L) ounces per day. In some embodiments, the OH⁻ solution is administered over the course of a day, for example the dosage is taken over eight hours, ten hours, twelve hours or 24 hours. In some embodiments, the dose may be calibrated based on body size, with effective doses comprising between about 0.01 to about 5 oz/pound, 0.3 to about 5 oz/pound, about 0.3 to about 3 oz/pound, about 0.3 to about 1 oz/pound, about 0.35 oz/pound. For example, an individual weighing 225 lbs would be given a starting dose of about 80 ounces (2.36 L) of diluted OH⁻ solution, as described in Example XI; an individual weighing 180 pounds would receive a starting dose of 64 oz (1.9 L) of the solution of Example XI per day. An individual weighing 135 pounds would receive a starting dose of 48 oz (1.4 L) of the solution of Example XI per day. An individual weighing 90 pounds would be given a starting dose of 32 oz (0.94 L) of the solution of Example XI per day. An individual weighing 45 pounds would be given a starting dose of 16 oz (0.47 L) of the solution of Example XI per day and an individual weighing 22 lbs would be given a starting dose of 8 oz (0.236 L) of the solution of Example XI per day. In other embodiments, fractions of the dosage are administered at particular time points, for example every hour, every two hours, every three hours, every four hours, every eight hours, every twelve hours, or any other fraction of time, as tolerated by the patient. For example, 0.5 L may be administered every hour, every two hours, every three hours, every four hours, every eight hours, every twelve hours, or any other fraction of time as tolerated by the patient. In one embodiment, one ounce of alkalizing material could be mixed in 1 liter of water. In another embodiment, three ounces of alkalizing material would be mixed in two liters of water. In a further embodiment, once the desired physiological pH level is obtained, a maintenance dose may be taken indefinitely. In some embodiments, a maintenance dose may be ½ of the therapeutic level, ⅓ of the therapeutic level, ¼ of the therapeutic level, or any other reduced dosage as determined by the judgment of the practitioner and the patient's circumstances.

The formulations may be presented in unit-dose or multi-dose containers.

Preferred unit dosage formulations are those containing a daily dose or unit, daily sub-dose, as described herein above, or an appropriate fraction thereof, of the active ingredient(s). In one embodiment eight ounces of the prepared formulation is administered every four hours. In another embodiment, eight ounces of the prepared formulation is administered every three hours. In a further embodiment, 0.5 L is administered every four hours. In another embodiment, 0.5 L is administered every eight hours. In still another embodiment, eight ounces of the prepared formulation is administered every two hours or fraction thereof. In exemplary embodiments, unit dose formulations are in 0.5 L, or a multiple thereof.

In other embodiments, fractions of the dosage are administered at particular time points, for example every hour, every two hours, every three hours, every four hours, every eight hours, every twelve hours, or any other fraction of time, as tolerated by the patient. In one embodiment, one ounce of anti-oxidant material could be mixed in 1 liter of water. In another embodiment, three ounces of anti-oxidant material would be mixed in two liters of water. In a further embodiment, once the desired physiological pH level is obtained, a maintenance dose may be taken indefinitely. In some embodiments, a maintenance dose may be ½ of the therapeutic level, ⅓ of the therapeutic level, ¼ of the therapeutic level, or any other reduced dosage as determined by the judgment of the practitioner and the patient's circumstances.

The formulations described herein may be manufactured and sold in a variety of forms. In some embodiments, they may be manufactured and sold as a single strength beverage for direct consumption by the consumer. In other embodiments, the formulations may be sold in an aqueous concentrate to be diluted with water to yield a beverage that treats or prevents acidosis, symptoms of acidosis, and conditions caused by or exacerbated by acidosis. Formulations may include excipients recognized in the art of pharmaceutical compounding including, but not limited to, binders, fillers, lubricants, emulsifiers, suspending agents, sweeteners, flavorings, preservatives, buffers, and other conventional excipients and additives. These additional formulation additives and agents will often be biologically inactive and can be administered to patients without causing deleterious side effects or interactions with the active agent.

The formulations may also be sold as a gel, powder, granule formation, or tablet which is to be dissolved in water to yield a beverage that treats or prevents acidosis, symptoms of acidosis, and conditions caused or exacerbated by acidosis.

In some embodiments, the compositions described herein are formulated for topical administration. Such topical administration may take the form of gels, lotions, milks, creams, water-in-oil or oil-in-water emulsions, sprays, suspensions, hair care products, and emollients. Such topical forms may include thickeners, stabilizers or gelling agents made from any suitable polysaccharide including, but not limited to, xanthan gum, carrageen, alginate, alginic acid, pectin, hyaluronic acid, chondroitin sulfate, selerorium, gum Arabic, gum karaya, gum tragacanth, carboxymethyl-chitin, cellulose gum, chitosan, cationic guar gum, hydroxyethylcellulose, starch, dextrins, guar gum, cellulose ethers, carboxymethylchitosan, N-hydroxy-dicarboxyethyl-chitosan, modified potato starch, cetyl hydroxyethylcellulose or polyquaternium. In some embodiments, the thickener may be an anionic polysaccharide, cationic polysaccharide, nonionic polysaccharide, amphoteric polysaccharide or hydrophobic polysaccharide.

In some embodiments, a suitable topical form such as a gel may comprise 1% to 10% xanthan gum or other thickening agent, preferably about 1% to about 5%, about 1% to about 3%, about 1% to about 2.9%, about 2.89% of the thickening agent by weight.

In some embodiments, the thickened alkaline water composition may additionally comprise a preservative such as preservative is ethyl alcohol, methyl alcohol, polyvinyl alcohol, and isopropyl alcohol. The preservative may comprise from about 1% to about 10%, about 2% to about 8%, about 3% to about 5%, about 2% to about 4%, about 3.785%, preservative such as, but not limited to, isopropyl alcohol by weight.

The thickened alkaline water composition is compatible with any type of emollient, preservative, pigment, vitamins, emulsifiers, ultraviolet filters and sunscreens, surfactants, preservatives, fragrance, humectants, glycols, oils, waxes, silicones, antioxidants or other agents generally used in formulations developed for topical application to the skin, hair or nails. In some embodiments, additional antioxidants may be added to the composition.

Any suitable emulsifier known in the art for use in water-in-oil or oil-in water or microemulsions may be used alone or in combination in the formulations described herein. Exemplary emulsifiers include, but are not limited to sesquioleates, ethoxylated esters of derivatives of natural oils, silicone emulsifiers, anionic emulsifiers, ethoxylated fatty alcohols, ethoxylated sorbitan esters, ethoxylated fatty acid esters, ethoxylated stearates, glyceryl monostearates, sorbitan esters, ethoxylated monoglycerides, ethoxylated diglycerides, ethoxylated triglycerides, methylglucose esters, polyacrylamide emulsifiers, polymeric emulsifiers, cationic emulsifiers, or mixtures thereof.

Any suitable emollient known to those of skill in the art may be used with the formulations and methods described herein. Exemplary emollients include, but are not limited to, hydrocarbon oils such as paraffin or mineral oils; waxes such as beeswax or paraffin wax; natural oils such as sunflower oil, apricot kernel oil, shea butter, or jojoba oil; silicone oils such as dimethicone, cyclomethicone or cetyldimethicone; fatty acid esters including, but not limited to, isopropyl palmitate, isopropyl myristate, dioctylmaleate, glyceryl oleate and cetostearyl isononanoate; fatty alcohols including, but not limited to, cetyl alcohol, or stearyl alcohol; polypropylene glycols; or polyethylene glycol ethers; and mixtures thereof.

Topical formulations as described herein may additionally include humectants or moisturizers including, but not limited to, polyols, exothylated glycerin, polyethylene glycols, propylene glycol, silicone glycol, xylitol, urea, honey, hydrogenated honey, hydrogenated starch hydrolystaes, glycerin, sorbitol, hydroxyethyl urea, 1,3-butylene glycol, aloe vera, and propylene glycol.

The gel formulations as described herein may be spread directly on mammalian skin or be applied to or part of a flexible support or pad which can then be applied to mammalian skin.

Additional OH⁻ solutions of the invention can be prepared and administered in any of a variety of inhalation or nasal delivery forms known in the art. Devices capable of depositing aerosolized OH⁻ formulations in the sinus cavity or pulmonary alveoli of a patient include metered dose inhalers, nebulizers, sprayers, and the like. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of calcium hydroxide or calcium oxide and any additional active or inactive ingredient(s).

Yet additional OH⁻ formulations are provided for parenteral administration, including aqueous and non-aqueous sterile injection solutions which may optionally contain antioxidants, buffers, bacteriostats and/or solutes which render the formulation isotonic with the blood of the mammalian subject; and aqueous and non-aqueous sterile suspensions which may include suspending agents and/or thickening agents.

In more detailed embodiments, OH⁻ compositions may be encapsulated for delivery in microcapsules, microparticles, or microspheres, prepared, for example, by coacervation techniques or by interfacial polymerization, in colloidal drug deliver) systems (for example, liposomes, albumin microspheres, microemulsions, nano-particles and nanocapsules) or in macroemulsions.

In further embodiments, the pharmaceutical agents of the invention may be administered parenterally, e.g. intravenously, intramuscularly, subcutaneously or intraperitoneally. The parenteral preparations may be solutions, dispersions or emulsions suitable for such administration. The subject agents may also be formulated into polymers for extended release following parenteral administration. Pharmaceutically acceptable formulations and ingredients will typically be sterile or readily sterilizable, biologically inert, and easily administered. Such polymeric materials are well known to those of ordinary skill in the pharmaceutical compounding arts.

The formulations described herein may be manufactured and sold in a variety of forms. In some embodiments, they may be manufactured and sold as a single strength beverage for direct consumption by the consumer. In other embodiments, the formulations may be sold in an aqueous concentrate to be diluted with water to yield a beverage that treats or prevents acidosis, symptoms of acidosis, and conditions caused by or exacerbated by acidosis. The formulations may also be sold as a powder, granule formation, or tablet which is to be dissolved in water to yield a beverage that treats or prevents acidosis, symptoms of acidosis, and conditions caused or exacerbated by acidosis. In other embodiments, the formulations described herein may be manufactured and sold in forms suitable for topical administration such as in the form of a lotion, gel, emollient, emulsion or cream.

In some embodiments, liquid formulations as described herein may be sold as part of a kit including a lactic acid meter, uric acid meter and/or pH test strips. The pH test strip would be effective between a pH of 4.5 and 9.0, with measurements in increments of 0.25. The kits of the present invention comprise one or more compositions of the present invention together with the lactic acid meter, uric acid meter and/or pH test strips, information which informs a user of the kit, by words, pictures, and/or the like, that use of the kit will provide one or more general health and/or general physiological benefits including, but not limited to, alkaline increasing, health and performance optimizing, illness preventing, energy level increasing, hydration increasing, recovery time decreasing, muscle protecting and stamina increasing benefits and which informs the user of the method of monitoring individual acidosis levels. By way of example only, the kit may comprise 7 bottles of 0.5 L of diluted alkaline water at a pH of 7.5. In other embodiments, the kit may comprise 7 bottles of concentrated alkaline water in 30 mL bottles at a pH of 12.5.

In other embodiments, the formulations described herein suitable for topical administration may be sold in a kit as part of a bandage or other flexible support in which the formulation is impregnated into the fibers.

The invention disclosed herein will also be understood to encompass methods and compositions comprising a base solution using in vivo metabolic products of the said compounds (either generated in vivo after administration of the subject precursor compound, or directly administered in the form of the metabolic product itself). Such products may result, for example, from the oxidation, reduction, hydrolysis, amidation, esterification and the like of the administered compound, primarily due to enzymatic processes. Accordingly, the invention includes methods and compositions of the invention employing compounds produced by a process comprising contacting a base solution of the present invention with a mammalian subject for a period of time sufficient to yield a metabolic product thereof.

The above disclosure generally describes the present invention. A more complete understanding can be obtained by referring to the following examples. These examples are described solely for purposes of illustration and are not intended to limit the scope of the invention. Although specific terms have been employed herein, such terms are intended for descriptive use and not for purposes of limitation.

### EXAMPLES

As demonstrated in the examples below, the present invention relates to the creation of a strong base solution for use as an antioxidant and/or alkalinity increasing agent.

### Example I

### Preparation of Basic Solution

50,000 g of Ca(OH)₂ is added to 500 gallons of water (100 g/gal) in a polyurethane tank surrounded by strong mono-polar magnets. The mixture is stirred until maximum disassociation is achieved. The solution is then passed through a 10 micron filter to remove any particulates. 78 ml of concentrated sulfuric acid (Baume 12°) per gallon, (39000 ml total) is added to a second polyurethane tank containing 500 gallons of pure water. The acid solution is circulated through an OzoTech OZ2PCS ozone generator (OzoTech, Inc., Yreka, Calif.) until the pH of the solution is above 7.0. The diluted sulfuric acid is then added to the filtered Ca(OH)₂ solution and the reaction is allowed to go to completion. The resulting solution is passed through a 10 micron filter to remove any anhydrous calcium sulfate.

### Example II

### Additional Purification of Ca(OH)₂ Solution

The solution of Example I is chilled to below 36° F. for up to four hours, but not allowed to freeze completely. The partially frozen material is then filtered using a 6 micron filter to remove any newly precipitated anhydrous calcium sulfate and/or ice. This increases the negative charge and the molar strength of the solution.

### Example III

### Preparation of an Antioxidant Solution

The solution of Example I is added to non-chlorinated drinking water and diluted until a pH of 8.5 to 9.0 is achieved.

### Example IV

### Treatment for Increasing Physiological pH

The solution of Example III is administered at the rate of 8 ounces every four hours until 24 to 32 ounces of the solution is consumed. Consumption of this amount increases physiological pH to normal levels and decreases rates of infection.

### Example V

### Treatment of Fungal Infections

Solutions of the basic solution of Example I, diluted to a pH of 11, were applied topically once a day to areas of tinea infection. The solution controlled the infection and prevented it from spreading.

### Example VI

### Preparation and Use of an Antioxidant Solution

An alcohol extraction of Dwarf Mistletoe, Arceuthobium campyopodum, was prepared to extract myricetin-3-0-galactoside and quercitin-3-0-galactoside. The berries of the Dwarf Mistletoe were harvested and then ground into a coarse powder. The powder was then placed in an Erlenmeyer flask with 80% cold methanol. After 24 hours, the methanol was decanted and saved, and a second aqueous extraction was carried out for a further 24 hours. The combined methanol eluents were evaporated under vacuum leaving an aqueous solution. A half ounce of the aqueous solution was then combined with 1 liter of the solution of Example I which had been diluted to a pH of 11. The resulting solution may then be taken over 8 hours.

### Example VII

### Preparation of Alkaline Water Solution Using Calcium Oxide

3.2 g of calcium oxide (CaO) was added to one liter of distilled or mineral free water. The mixture was stirred for approximately ten minutes and filtered with a non-charcoal five micron filter resulting in water with a total alkalinity of 2000 mg/L CaCO₃.

### Example VIII

### Preparation of Extra Strength Alkaline Water Solution Using Calcium Oxide

4 grams of calcium oxide (CaO) were added to one liter of distilled or mineral free water. The mixture was stirred for approximately ten minutes and then filtered with a non-charcoal five micron filter resulting in water with a 2400 mg/L CaCO₃ total alkalinity.

### Example IX

### Preparation and Use of Concentrated Calcium Oxide Based Alkaline Water Solution

1 oz of the concentrate of Example VIII or VIII was diluted in 32 oz of purified, demineralized or distilled water. A mammalian subject consumes 8 ounces every four hours until 24 to 32 ounces of the solution is consumed. Consumption of this amount increases physiological pH to normal levels and decreases rates of infection.

### Example X

### Preparation of Concentrated Alkaline Water Using Calcium Oxide-Version 2

Five gallons of filtered water are added to a non-reactive drum fitted with a bucket mixer. While stirring, calcium oxide is added until the solution reaches a pH of 12.75 pH as determined by a Waterproof EcoTestr pH 2 (Oakton Instruments, Vernon Hills, Ill.) and has a conductivity of between 700 μS/cm to 750 μS/cm as determined by COM-100: Waterproof EC/TDS/Temp Combo Meter (HM Digital, Inc., Culver City, Calif.). The resulting solution is then filtered with a non-charcoal live micron filter and decanted into 30 mL containers for distribution.

### Example XI

### Dilution of Alkaline Water for Consumption

Five gallons of filtered water are added to a non-reactive drum fitted with a bucket mixer. While stirring, calcium oxide is added until the solution reaches a pH of 12.75 pH as determined by a Waterproof EcoTestr pH 2 (Oakton Instruments, Vernon Hills, Ill.) and has a conductivity of between 700 μS/cm to 750 μS/cm as determined by COM-100: Waterproof EC/TDS/Temp Combo Meter (HM Digital, Inc., Culver City, Calif.). 30 mL of the resulting solution is put in a second non-reactive drum fitted with a bucket mixer and add water. The resulting solution has a pH of 7 as determined by a Waterproof EcoTestr pH 2 (Oakton Instruments, Vernon Hills, Ill.) and has a conductivity of 50 μS as determined by COM-100: Waterproof EC/TDS/Temp Combo Meter (HM Digital, Inc., Culver City, Calif. The solution is filtered with a non-charcoal five micron filter and then decanted into 16.9 oz (0.5 L) containers for consumption.

### Example XII

### Preparation of Alkaline Water Using Sodium Hydroxide

Five gallons of filtered water are added to a non-reactive drum fitted with a bucket mixer. While stirring, sodium hydroxide is added until the solution reaches a pH of 12.5 pH as determined by a Waterproof EcoTestr pH 2 (Oakton Instruments, Vernon Hills, Ill.) and has a conductivity of between 700 μS/cm to 750 μS/cm as determined by COM-100: Waterproof EC/TDS/Temp Combo Meter (HM Digital, Inc., Culver City, Calif.).

### Example XIII

### Preparation of Alkaline Water Using Calcium Oxide—Version 3

In a 5 gallon non-reactive drum fitted with a mixing device, 9.5 g of calcium oxide is added to 18.9 liters of distilled water and thoroughly mixed at 26.6° C. The pH of the resulting mixture is measured using a Waterproof EcoTestr pH 2 (Oakton Instruments, Vernon Hills, Ill.). If the pH is less than 12.5, calcium oxide is added by milligrams until the desired pH is achieved. The conductivity of the resulting solution is then measured using a COM-100: Waterproof EC/TDS/Temp Combo Meter (HM Digital, Inc., Culver City, Calif.) and is between 700 μS/cm and 750 μS/cm.

### Example XIV

### Preparation of Concentrated Alkaline Water Using Calcium Oxide-Version 4

In a 1.5 glass liter beaker, 500 mg of calcium oxide is added to 1 liter of water and mixed thoroughly at 26.6° C. The pH of the resulting mixture is measured using a Waterproof EcoTestr pH 2 (Oakton Instruments, Vernon Hills, Ill.). If the pH is less than 12.5, calcium oxide is added by milligrams until the desired pH is achieved. The conductivity of the resulting solution is then measured using a COM-100: Waterproof EC/TDS/Temp Combo Meter (HM Digital, Inc., Culver City, Calif.) and is between 700 and 750 μS.

### Example XV

### Alkaline Gel

9.5 g of calcium oxide were added to 18.9 liters of distilled water in a sterilized mixing container and stirred thoroughly. To the resulting suspension was added 591.5 g of xanthan gum and blended until smooth. Then 1 liter of 98% isopropyl alcohol and 28.35 g of lemon oil were added and mixed well. The resulting mixture was then packaged in 0.23 L (8 oz) sterile containers.

### Example XVI

### Treatment of Nail Infections

Nails are soaked in a solution of 1 oz of the alkaline water solution of Example X mixed with one ounce of water for thirty minutes once a day. The nails are then coated with alkaline gel of Example XV.

### Example XVII

### Orange Gel

9.5 g of calcium oxide were added to 18.9 liters of distilled water in a sterilized mixing container and stirred thoroughly. To the resulting suspension was added 591.5 g of xanthan gum and blended until smooth. Then 1 liter of 98% isopropyl alcohol and 28.35 g of organic orange oil were added and mixed well. The resulting mixture was then packaged in sterile containers.

### Example XVIII

### Treatment of MRSA

Methicillin-resistant Staphylococcus aureus is a bacterium responsible for several difficult-to-treat infections in humans. It generally causes skin infections similar to boils, but can also infect the blood stream, lungs, or the urinary tract. For a skin infection, Alkaline gel of Example XV or XVII is liberally applied to the site of infection at least three times per day until the infection is resolved.

### Example XIX

### Determination of Effectiveness of Alkaline Water

Ten (10) healthy male subjects ranging from 25 to 35 years old are given 2 L of water in 0.5 L doses as a placebo for three days. They are then given 2 L of Alkaline water in 0.5 L doses every four hours as prepared in Example XI daily for two weeks. On days three, seventeen, and eighteen the subjects undergo aerobic performance assessment on a stationary exercise bicycle in which power is increased from 25 watts to 175 watts at 25 watt intervals. Each interval lasts three minutes. At the end of each interval, peripheral blood is collected to measure lactic acid and anaerobic tolerance. Peripheral blood is collected at 5 minutes, 30 minutes, 1 hour and 24 hours after the last interval. Subjects have continuous heart rate VO₂ and CO₂ monitoring. Lactic acid levels are measured using a Lactic Acid meter (Sports Resource Group (Hawthorne, N.Y.)).

### Example XX

### Determination of Change in Lactic Acid Threshold

Ten (10) healthy males participate in this trial. Subjects warm up for 15 minutes on a stationary bike and then work to their peak sustained intensity within the first 10 minutes and continue for twenty minutes. Using a heart rate monitor, the average heart rate is calculated over the last 20 minutes. Each subject is then given 2 L of Alkaline water in 0.5 L doses taken four times a day as prepared in Example XI daily for two weeks. After two weeks, the subjects are retested and the average heart rate (estimated heart rate at subject's lactate threshold) is compared.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited with the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

### REFERENCES

Bizzel, Lorrie, Respiratory Support Lucile Packard Children's Hospital Heart Center/CVICUhttp://lane.stanford.edu/portals/cvicu/HCP_Respiratory-Pulmoanry_Tab_{—}2/Mechanisms_of_Support.pdf (March, 2009)
Brandis, Kerry, Acid-base pHysiology’ http://www.anaesthesiaMCQ.com
Bryan S. Judge, MD, Metabolic Acidosis: Differentiating the Causes in the Poisoned Patient. Med Clin N Am 89 (2005) 1107-1124.
Garrett, William E. and Donald T. Kirkendall, Exercise and Sport Lippincott Williams & Wilkins; 1st edition (Jan. 15, 2000) p. 61.
Kubera B, Hubold C, Otte S, Lindenberg A S, Zeiss I, Krause R, Steinkamp M, Klement J, Entringer S, Pellerin L, Peters A. Rise in plasma lactate concentrations with psychosocial stress: a possible sign of cerebral energy demand. Obes Facts. 2012; 5(3):384-92. doi: 10.1159/000339958. Epub 2012 June 22.
McArdle, W. D., Katch, F. I., & Katch. V. L. 1996. Exercise Physiology: Energy, Nutrition, and Human Performance. Baltimore, Md.: Williams & Wilkins.
Mulkey, Daniel K., Henderson, Richard A., Ritucci, Nick A. Putnam, Robert W., Deam Jay B. Oxidative stress decreases pHi and Na_/H_ exchange and increases excitability of solitary complex neurons from rat brain slices. Am J Physiol Cell Physiol 286: C940-C951, 2004.
Robergs, R. A., Ghiasvand, F., Parker, D. (2004). Biochemistry of exercise-induced metabolic acidosis. American Journal of Physiology: Regulatory, Integrative and Comparative Physiology. 287: R502-R516.
Robergs, R. A., & Roberts, S. 1997. Exercise Physiology: Exercise, performance, and clinical applications. St Louis, Mo.: Mosby.
Seifter J L. Acid-base disorders:chap 120. In: Goldman L, Schafer Al, eds. Cecil Medicine. 24th ed. Philadelphia, Pa.: Saunders Elsevier; 2011
Strohle A, Hahn A, Sebastian, A: Estimation of the diet-dependent net acid load in 229 worldwide historically studied hunter-gatherer societies. Am J Clin Nutr 91: 406-412, 2010.
Torpy, Janet M. MD; Cassio Lynm, MA; Richard M. Glass, MD Chronic Stress and the Heart JAMA. 2007; 298(14):1722.
Vrijkotte T G, van Doornen L J, de Geus E J. Effects of work stress on ambulatory blood pressure, heart rate, and heart rate variability. Hypertension. 2000 April; 35(4):880-6.

We claim:

1. A method for preparing a resultant mixture having a high concentration of OH− ions comprising:
a) preparing a first solution by adding calcium oxide to water;
b) agitating the first solution to increase a rate or amount of dissociation of the calcium oxide;
c) measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5.

2. The method of claim 1, wherein the second solution has a conductivity from about 700 μS/cm to about 2000 μS/cm.

3. The method of claim of claim 1, wherein the second solution has a conductivity from about 700 μS/cm to about 750 μS/cm.

4. A method for preparing a resultant mixture having a high concentration of OH− ions comprising:
a) preparing a first solution by adding calcium oxide to water;
b) agitating the first solution to increase a rate or amount of dissociation of the calcium oxide;
c) measuring the pH of the first solution; wherein if the pH of the solution is less than 12.5, an additional amount of calcium oxide is added to the first solution to create a second solution;
d) agitating the second solution to increase a rate or amount of dissociation of the calcium oxide;
e) measuring the pH of the second solution to determine that it has a pH of about 12.5; and
f) diluting the second solution with water so that it has a pH of about 7.5.

5. The method of claim 4, wherein the resultant mixture has a conductivity of about 60 μS/cm

6. A method of increasing alkalinity in a mammalian subject suffering from acidosis comprising:
a) preparing a first solution by adding calcium oxide to water;
b) agitating the first solution to increase a rate or amount of dissociation of the calcium oxide;
c) measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5.
f) diluting the second solution to a pH of about 7.5 to produce a diluted resultant mixture; and
g) administering an alkalinity increasing amount of the diluted resultant mixture to the mammalian subject.

7. The method of claim 6, wherein the diluted resultant mixture has a conductivity of about 60 μS/cm.

8. The method of claim 6, wherein the alkalinity increasing amount comprises between about 1 to about 2 liters of the composition per day.

9. The method of claim 6, wherein the alkalinity increasing effective amount comprises about 0.5 liters every eight hours.

10. A method of lowering serum lactic acid levels comprising administering an effective amount of an alkalizing composition in 0.5 L servings wherein a 0.5 liter serving comprises:
0.5 liters of a dilution to a pH of 7.5 of a first solution prepared by adding between about 500 mg to about 600 mg of calcium oxide to a liter of water and agitating the first solution to increase a rate or amount of dissociation of the calcium oxide.

11. The method of claim 10, wherein an effective amount lowers serum lactic acid levels by about 0.5 to about 0.1 mmol/L.

12. The method of claim 10, wherein an effective amount of an alkalizing composition lowers serum lactic acid levels by about 0.25 to about 0.5 mmol/L.

13. A method of treating systemic inflammatory response syndrome comprising administering to a mammalian subject an effective amount of an alkalizing composition prepared by:
a) creating a first solution by adding calcium oxide to water;
b) agitating the first solution to increase a rate or amount of dissociation of the calcium oxide;
c) measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5;
f) diluting the second solution to a pH of about 7.5 to produce a diluted resultant mixture; and
g) administering and effective amount of the diluted resultant mixture to the mammalian subject.

14. The method of claim 13, wherein the diluted resultant mixture has a conductivity of about 60 μS/cm.

15. The method of claim 13, wherein the effective amount of the alkalizing composition comprises between about 1 to about 2 liters of the composition per day.

16. The method of claim 13, wherein the effective amount of the alkalizing composition comprises about 0.5 liters every four hours.

17. A method of treating hyperlactemia comprising administering to a mammalian subject an effective amount of an alkalizing composition prepared by
a) creating a first solution by adding calcium oxide to water;
b) agitating the first solution to increase a rate or amount of dissociation of the calcium oxide;
c) measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5;
f) diluting the second solution to a pH of about 7.5 to produce a diluted resultant mixture; and
g) administering and effective amount of the diluted resultant mixture to the mammalian subject.

18. The method of claim 17, wherein the diluted resultant mixture has a conductivity of about 60 μS/cm.

19. The method of claim 17, wherein the alkalinity increasing amount comprises between about 1 to about 2 liters of the diluted resultant mixture per day.

20. The method of claim 17, wherein the alkalinity increasing effective amount comprises about 0.5 liters of the diluted resultant mixture every four hours.

21. The method of claim 17, wherein the alkalinity increasing effective amount is effective to decrease serum lactate levels by about 0.5 to 1.0 mmol/L.

22. The method of claim 17, wherein the alkalinity increasing effective amount is effective to decrease serum lactate levels by about 0.25 to 0.5 mmol/L.

23. A method of treating methicillin-resistant Staphylococcus aureus comprising administering to a mammalian subject an effective amount of an alkalizing composition prepared by:
a) creating a first solution by adding calcium oxide to water;
b) agitating the first solution to increase a rate or amount of dissociation of the calcium oxide;
c) measuring the pH of the first solution; wherein if the pH of the solution is less than 12.5, an additional amount of calcium oxide is added to the first solution to create a second solution;
d) agitating the second solution to increase a rate or amount of dissociation of the calcium oxide; and
e) measuring the pH of the second solution to determine that it has a pH of about 12.5;
f) diluting the second solution to a pH of about 7.5 to produce a diluted resultant mixture; and
g) administering an effective amount of the diluted resultant mixture to the mammalian subject.

24. The method of claim 23, wherein the diluted resultant mixture has a conductivity of about 60 μS/cm.

25. The method of claim 23, wherein the alkalinity increasing effective amount comprises between about 1 to about 2 liters of the composition per day.

26. The method of claim 23, wherein the alkalinity increasing effective amount comprises about 0.5 liters every four hours.

27. An alkalizing gel for topical application comprising, based on the total weight,
80-95% water;
0.01-0.09% CaO;
1-4% thickener; and
2-5% preservative; and
wherein the sum of the percentages is equal to 100%.

28. The alkalizing gel of claim 27, wherein the thickener is xanthan gum, carrageen, alginate, alginic acid, pectin, hyaluronic acid, chondroitin sulfate, gum Arabic, selerorium, gum karaya, gum tragacanth, carboxymethyl-chitin, cellulose gum, chitosan, cationic guar gum, hydroxyethylcellulose, starch, dextrins, guar gum, cellulose ethers, carboxymethylchitosan, N-hydroxy-dicarboxyethyl-chitosan, modified potato starch, cetyl hydroxyethylcellulose or polyquaternium.

29. The alkalizing gel of claim 28, wherein the thickener is xanthan gum.

30. The alkalizing gel of claim 27, wherein the preservative is ethyl alcohol, methyl alcohol and isopropyl alcohol.

31. The alkalizing gel of claim 27, further comprising 0.05%-0.3% fragrance.

32. The alkalizing gel of claim 27, wherein the fragrance is lemon oil.

33. The alkalizing gel of claim 27, wherein the fragrance is orange oil.

34. A method of treating a methicillin-resistant Staphylococcus aureus infection on the skin of a mammalian subject comprising applying an effective amount of an alkalizing gel to the site of the infection, wherein the alkalizing gel comprises based on the total weight,
80-95% water;
0.01-0.09% CaO;
1-4% thickener; and
2-5% preservative;
wherein the sum of the percentages is equal to 100%.

35. The alkalizing gel of claim 31, wherein the thickener is xanthan gum, carrageen, selerorium, alginate, alginic acid, pectin, hyaluronic acid, chondroitin sulfate, gum Arabic, gum karaya, gum tragacanth, carboxymethyl-chitin, cellulose gum, chitosan, cationic guar gum, hydroxyethylcellulose, starch, dextrins, guar gum, cellulose ethers, carboxymethylchitosan, N-hydroxy-dicarboxyethyl-chitosan, modified potato starch, cetyl hydroxyethylcellulose or polyquaternium.

36. The alkalizing gel of claim 31, wherein the thickener is xanthan gum.

37. The alkalizing gel of claim 31, wherein the preservative is ethyl alcohol, methyl alcohol and isopropyl alcohol.

38. The alkalizing gel of claim 31, further comprising 0.05-0.2% fragrance.

39. The alkalizing gel of claim 31, wherein the fragrance is lemon oil.

40. The alkalizing gel of claim 31, wherein the fragrance is orange oil.

41. The method of claim 31, wherein the alkalizing gel is on a flexible support.

42. The method of claim 41, wherein the flexible support is a bandage.
